(19) United States
(12) Patent Application Publication
(10) Pub. No.: US 2009/0220429 A1
(43) Pub. Date: Sep. 3, 2009

(54) METHOD AND A SYSTEM FOR ASSESSING NEUROLOGICAL CONDITIONS

(75) Inventors: Kristinn Johnsen, Reykjavik (IS); Gisli Holmar Johannesson, Gardabaer (IS); Steinn Gudmundsson, Reykjavik (IS); Johannes Helgason, Akranes (IS)

Correspondence Address: KNOBBE MARTENS OLSON & BEAR LLP, 2040 MAIN STREET, FOURTEENTH FLOOR, IRVINE, CA, 92614, US

(73) Assignee: METIS CURA EHF, REYKJAVIK (IS)

(21) Appl. No.: 11/817,780

(22) PCT Filed: Mar. 3, 2006
(86) PCT No.: PCT/EP06/02157

§ 371 Date: Jul. 18, 2008

(30) Foreign Application Priority Data
Mar. 4, 2005 (IS) IS 7727

(51) Int. Cl. A61B 5/055; A61B 5/0476; A61K 49/00; A61K 49/04

(52) U.S. Cl. 424/9.3; 600/544; 600/411; 424/9.1; 424/9.4

(57) ABSTRACT

This invention relates to a method and a system for generating a discriminatory signal for a neurological condition, where at least one probe compound that has a neurophysiologic effect is provided. Biosignal data are obtained from a subject based on biosignal measurements obtained from biosignal measuring device adapted for placement on a subject, wherein said biosignal data are obtained posterior to the administering of said probe compound to the subject. Analogous biosignal reference data are provided for reference subjects in at least one reference group posterior to the administering of the probe compound, wherein the reference data are utilized for defining reference features having common characteristics between the reference subjects in the at least one reference group, wherein the reference data are processed for defining reference posterior probability vectors for each respective reference subject, wherein each respective posterior probability vector comprises particular feature or a feature combination elements with probability values associated to said elements, the posterior probability vectors resulting in a distribution of said features or feature combinations for said reference subjects. Subsequently, the biosignal data obtained from the subject are used for calculating analogues posterior probability vector for said subject. The discriminatory signal is then generated based on comparison between said posterior probability vector for said subject and the distribution of said features or feature combinations.

### FIELD OF THE INVENTION

The present invention relates to a method and a system for generating a discriminatory signal for a neurological condition by making use of a solid reference data obtained from reference subjects.

### BACKGROUND OF THE INVENTION

US2003/0233250 discloses a method for providing a data interpretation tool for biological data associated with a patient via a network, where biological data of the patient are collected and a portion of the data is transmitted over the network to a storage device. At least one potential indicator variable associated with the patients biological data is then determined and compared to standardized set of data associated with the health condition. Based upon the comparison, at least one indicator variable is selected and a report is generated including the indicator variable and at least one data interpretation tool to a health care provider associated with the patient.

In the report evaluation scheme, the reports and Indicators used for characterizing conditions are kept up to date with current scientific knowledge. To this end, the standardized set of data comprises data collected by staff of professionals, where the data are obtained from relevant research articles (0076, 0074) and in house database, where these data are used to uncover new indicator variables for a particular condition. It follows that new Indicators are collected based on the evaluation of the data. The report formats are revised based on newly developed indicators that are used to create the data interpretation tools.

Just the fact that the standardized set of data is defined by the professionals i.e. defined manually causes that these data may not be precise enough to be used as reference data. Furthermore, this reference does not state how, where, when or under which conditions the data are collected. It might be essential that the biological data to be used as standardized set of data (reference data) are collected in precisely the same manner as patient's biological data, and that the people providing these reference data fulfill certain requirements, e.g. age, gender etc. However, this reference only states that the data are obtained from research articles and in-house database. This can easily result in that the reference data are not reliable enough to provide a convincing diagnosing. Considering early diagnosing, it is essential that the reference data are very well defined, since the deviation between the patients biological data and the reference data can be extremely small and not detectable if the reference data are not well enough defined.

It is also clear from this reference that it is not oriented towards diseases where a stimulus is needed in order to initiate a certain reaction that segregates a subject from a diseased subject. This problem is however partly solved in Holscneider et. al. (“Attenuation of brain high frequency electrocortical response after thiopental in early stages of Alzheimer's dementia”, Psychopharmacology (2000), 149: 6-11) which discloses a method of detecting whether at an early stage the phenomenon relating to a loss of high-frequency (beta) brain electrical responses to thiopental in dementia Alzheimer's (DAT) is detectable. The result presented in this reference showed that no significant group difference in beta power was detectable at the baseline, but in response to thiopental, early DAT subjects compared to controls showed a significantly smaller beta power response in the frontal region at 1-3 min post-injection. Accordingly, the drug thiopental can be used as a stimulus to cause a trend that is measurable between objects suffering from DAT and healthy objects.

However, this reference merely discloses the concept of how two discriminate between diseased subjects suffering from DAT and healthy subjects by using the drug thiopental, wherein in the absence of the drug such discrimination would not be possible.

The reference does however not disclose how to disclose how to distinguish a subject from a reference subject at an early stage.

There is therefore a great need for effective and accurate and effective diagnostic method for early diagnosing of diseases and conditions of the central nervous system such as Alzheimer's disease and other neurodegenerative diseases as well as mental conditions.

### SUMMARY OF THE INVENTION

Accordingly, the present invention overcomes the above mentioned problems by providing a method and a system that enable an early diagnosing of subjects suffering form neurological disease by making use of highly accurate reference data obtained from carefully selected reference subjects.

According to one aspect, the present Invention relates to a method of generating a discriminatory signal for a neurological condition comprising:

providing at least one probe compound that has a neurophysiologic effect,
obtaining biosignal data from a subject based on biosignal measurements obtained from biosignal measuring device adapted for placement on a subject, wherein said biosignal data are obtained posterior to the administering of said probe compound to the subject,
providing analogous biosignal reference data for reference subjects in at least one reference group posterior to the administering of said probe compound, wherein the reference data are utilized for defining reference features having common characteristics between the reference subjects in said at least one reference group, wherein said reference data are processed for defining reference posterior probability vectors for each respective reference subject, wherein each respective posterior probability vector comprises particular feature or a feature combination elements with probability values associated to said elements, said posterior probability vectors resulting in a distribution of said features or feature combinations for said reference subjects,
utilizing said biosignal data from said subject for calculating analogues posterior probability vector for said subject,
wherein said discriminatory signal is generated based on comparison between said posterior probability vector for said subject and the distribution of said features or feature combinations.

It is clear that by generating a reference in such a statistical way, a very solid background data is provided which is essential for allowing a determination of said discriminatory signal at an early stage of a neurological disease. Also, since the determination of said posterior probability vector follows the posterior probability vectors of the reference subject, the current condition of the subject can be compared precisely with said distribution of the posterior probability vectors of said reference subject. This can be explained more clearly with a simplified example. A possible feature combination for features f1, f2 and f3 (f1 could be the relative theta power, f2 could be the relative alpha power and f3 could be the spectral entropy) could be [(f1,f2);(f1,f3);(f2,f3)]. By plotting such a feature combination into three different diagrams, the first diagram representing the (f1,f2), the second diagram the (f1,f3) combination and the third diagram the third combination (f2,3), for all the reference subjects, a distribution of said feature pairs is obtained. Accordingly, the posterior probability vectors for each respective reference person comprises e.g. the probability with respect to e.g. group B that subject within domain A is classified in said domain, e.g. P=[0.9, 0.87, 0.32] indicates that for feature pair (f1,f2) and (f1,f3) the probability that the subject is within domain is high. However, for feature pair (f2,f3) the probability is low. Such a posterior probability vector could be implemented in the way that the first two elements having such a high variance are to be used as good distribution “candidates”, whereas the last element in the posterior probability vector is to be neglected.

Accordingly, it follows that such a discriminatory signal can be generated at a very early stage of the disease and used for diagnosing the subject. Clearly, such an early diagnosis can be essential for the subject. It is furthermore clear from the present invention that the implementation of said compound is to cause a trend between the obtained biosignal data between the subject and the group of the reference subjects, or enhance this trend. This can only result in a better discrimination between the diseased subject and a healthy subject. Additionally, the possibility of using more than one reference feature values in generating said statistical model can only result in enhanced accuracy of the discriminatory signal and therefore more reliable diagnosis. The term subject means according to the present invention a human being, but the term can just as well relate to animals and other biological organism.

In an embodiment, said method further comprises obtaining biosignal data from said subject and said reference subjects prior to administering said probe compound. This can be of particular advantage since these data can be used as reference data, e.g. by subtracting said data from the data obtained posterior to the administering of the probe compound. Furthermore, the data can be used as additional information source for determining reference features. Accordingly, one feature results in multiplicity of features, e.g. a feature prior and feature subsequent resulting in e.g. f1(prior)-f(posterior); f1(posterior)-f1(prior); f1(posterior)/f(prior); f1(prior)/f1(posterior) etc. This gives a multiplicity of features

In an embodiment, the method further comprises selecting out only those elements in said reference posterior probability vectors that have variance value above a pre-defined threshold value. This is of particular importance when generating so-called feature extraction, meaning that only those elements in the posterior probability vector that have variance above a certain threshold value are to be selected as candidates. Assuming that for a subject in group A that has a posterior probability vector P[1.0, 0.85, 0.25] for a subject within group A, it is clear that from the first element (could e.g. be said (f1,f2) feature combination) a perfect trend is found for the first element (i.e. 100% possibility that the subject is within domain A, this indicates a perfect trend between e.g. groups A and B, i.e. there is no overlap), a very high trend for the second element (85% possibility that the subject is within domain A for e.g. the (f1,f2) feature combination), but for the last element there are only 25% possibility that the subject lies within group A. This last element indicates that subject A lies within groups B or close to the boundaries between group A and B. Accordingly, the first two elements have high variance, and the last element a low variance. If the threshold value is e.g. 0.6, the last element would be eliminated and the first two element would be used as candidates for said posterior probability vector distribution for the reference subject. The result thereof is a so-called feature extraction which will generate a clear trend between groups A and B as an example. This means that said two groups will be fully separated, meaning that two groups of different properties are created. Accordingly, if the result of a subject indicates that the subject lies within group A (the elements of the posterior probability vector lies within group A as an example) the subject has common properties with the subject of said group.

In an embodiment, said one or more biosignal measurements comprise an electroencephalographic (EEG) measurement.

In an embodiment, the neurological condition is selected from the group consisting of Alzheimer's disease, multiple schlerosis, mental conditions including depressive disorders, bipolar disorder and schizophrenic disorders, Parkinsons' disease, epilepsy, migraine, Vascular Dementia (VaD), Fronto-temporal dementia, Lewy bodies dementia, Creutzfeld-Jacob disease and vCJD (“mad cow” disease).

In an embodiment, said one or more biosignal measurements comprise a biosignal measurement selected from the group consisting of magnetic resonance imaging (MRI), functional magnetic resonance imaging (FMRI), magnetoencephalographic (MEG) measurements, positron emission tomography (PET), CAT scanning (Computed Axial Tomography) and single photon emission computerized tomography (SPECT).

In an embodiment, said at least one probe compound is selected from the group consisting of compounds from the group of consisting of GABA affecting drugs including propofol and etomidate; barbiturates including methohexital, thiopental, thiamylal, buthalital, thialbarbital, hexobarbital, pentobarbital, secobarbital, hexethal, butalbital, cyclobarbital, talbutal, phenobarbital, mephobarbital, and barbital; benzodiazepines such as alprazolam, bromazepam, chlordiazepoxide, clobazam, clonazepam, clorazepate, clozapine, olanazapine diazepam, estazolam, flunitrazepam, flurazepam, halazepam, ketazolam, loprazolam, lorazepam, lormetazepam, medazepam, midazolam, nitrazepam, nordazepam, oxazepam, prazepam, ouazepam, temazepam, and triazolam; cholinergic agonists such as aceclidine, AF-30, AF150, AF267B, alvameline, arecoline, bethanechol, CDD-0102, CDD-0034-C, CDD-0097-A, cevimeline, CI 1017, cis-dioxolane, milameline, muscarine, oxotremorine, pilocarpine, RS86, RU 35963, RU 47213, sabcomeline, SDZ-210-086, SR 46559A, talsaclidine, tazomeline, UH5, xanomellne, and YM 796; cholinergic antagonists including AF-DX 116, anisotropine, aprophen, AQ-RA 741, atropin, belladonna, benactyzine, benztropine, BIBN 99, DIBD, cisapride, clidinium, darifenacin, dicyclomine, glycopyrrolate, homatropine, atropine, hyoscyamine, ipratroplum, mepenzolate, methantheline, methscopolamine, PG-9, pirenzepine, propantheline, SCH-57790; SCH-72788, SCH-217443, scopolamine, tiotropium, tolterodine, and trihexyphenidyl; acetyl choline esterase (ACE) inhibitors including 4-aminopyridine, 7-methoxytacrine, amiridine, besipirdine, CHF2819, CI-1002, DMP 543, donepezil, eptastigmine, galantamine, huperzine A, huprine X, huprine Y, MDL 73745, metrifonate, P10358, P11012, phenserine, physostigmine, ouilostigmine, rivastigmine, Ro 46-5934, SM-10888, suronacrine, T-82, tacrine, TAK-147, tolserine, trifluoroacetophenone, TV3326, velnacrine, and zifrosilone; ACh release enhancers including linopirdine, and XE991; Choline uptake enhancers including MKC-231, and Z-4105; nicotinic agonists including: ABT-089, ABT-418, GTS-21, and SIB-1553A; NMDA antagonist including ketamine, and memantine; serotonin inhibitor such as cinanserin hydrochloride, fenclonine, fonazine mesylate, and xylamidine tosylate; serotonin antagonist including altanserin tartrate, aAmesergide, cyproheptadiene, granisetron, homochlorcyclizine, ketanserin, mescaline, mianserin, mirtazapine, perlapine, pizotyline, olanzapine, ondansetron, oxetorone, risperidone, ritanserin, tropanserin hydrochloride, and zatosetron; serotonin agonists including 2-methylserotonin, 8-hydroxy-DPAT, buspirone, gepirone, ipsapirone, rizatriptan, sumatriptan, and zolmatriptan; serotonin reuptake inhibitors including citalopram, escitalopram oxalate, fluoxetine, fluvoxamine, paroxetine, and sertraline; dopamine antagonists including pimozide, ouetiapine, metoclopramide, and dopamine precursors including levodopa.

In an embodiment, said one or more biosignal measurements comprise an electroencephalographic (EEG) measurement.

In an embodiment, said two or more compounds are used for stimulating two or more different neurophysiologic effects. This can be of particular importance for determining whether a subject lies within different groups or not. Clearly, different groups have different characteristics. Therefore, it is highly relevant of obtain features that represent said different characteristics.

In an embodiment, said method further comprises subjecting the subject to a sensory stimulus prior to or during the biosignal measurement. This can be of particular importance for initiating a certain reaction that segregates a subject from the reference subjects.

In an embodiment, said features are selected from a group consisting of the absolute delta power, the absolute theta power, the absolute alpha power, the absolute beta power, the absolute gamma power, the relative delta power, the relative theta power, the relative alpha power, the relative beta power, the relative gamma power, the total power, the peak frequency, the median frequency, the spectral entropy, the DFA scaling exponent (alpha band oscillations), the DFA scaling exponent (beta band oscillations) and the total entropy.

According to another aspect, the present invention relates to a computer readable media for storing instructions for enabling a processing unit to execute the above method steps.

According to yet another aspect, the present invention relates to a system adapted for generating a discriminatory signal for a neurological condition of a subject posterior to administering at least one compound that has a neurophysiologic effect comprising:

a receiver unit for receiving biosignal data for a subject from biosignal measuring device after administering said at least one compound,
an internal or external storage means for storing analogous biosignal reference data for reference subjects in at least one reference group posterior to the administering of said probe compound, wherein the reference data are utilized for defining reference features having common characteristics between the reference subjects in said at least one reference group, wherein said reference data are processed for defining reference posterior probability vectors for each respective reference subject, wherein each respective posterior probability vector comprises particular feature or a feature combination elements with probability values associated to said elements, said posterior probability vectors resulting in a distribution of said features or feature combinations for said reference subjects, a processor for utilizing said biosignal data from said subject for calculating analogues posterior probability vector for said subject, said processor being adapted for generating said discriminatory signal based on comparison between said posterior probability vector for said subject and the distribution of said features or feature combinations.

According to still another aspect, the present invention relates to the use of at least one compound selected from a group consisting of GABA affecting drugs including propofol and etomidate; barbiturates including methohexital, thiopental, thiamylal, buthalital, thialbarbital, hexobarbital, pentobarbital, secobarbital, hexethal, butalbital, cyclobarbital, talbutal, phenobarbital, mephobarbital, and barbital; benzodiazepines such as aiprazolam, bromazepam, chlordiazepoxide, clobazam, clonazepam, clorazepate, clozapine, olanazapine diazepam, estazolam, flunitrazepam, flurazepam, halazepam, ketazolam, loprazolam, lorazepam, lormetazepam, medazepam, midazolam, nitrazepam, nordazepam, oxazepam, prazepam, ouazepam, temazepam, and triazolam; cholinergic agonists such as aceclidine, AF-30, AF150, AF267B, alvameline, arecoline, bethanechol, CDD-0102, CDD-0034-C, CDD-0097-A, cevimeline, CI 1017, cis-dioxolane, milameline, muscarine, oxotremorine, pilocarpine, RS86, RU 35963, RU 47213, sabcomeline, SDZ-210-086, SR 46559A, talsaclidine, tazomeline, UH5, xanomeline, and YM 796; cholinergic antagonists including AF-DX 116, anisotropine, aprophen, AQ-RA 741, atropin, belladonna, benactyzine, benztropine, BIBN 99, DIBD, cisapride, clidinium, darifenacin, dicyclomine, glycopyrrolate, homatropine, atropine, hyoscyamine, ipratropium, mepenzolate, methantheline, methscopolamine, PG-9, pirenzepine, propantheline, SCH-57790; SCH-72788, SCH-217443, scopolamine, tiotroplum, tolterodine, and trihexyphenidyl; acetyl choline esterase (ACE) inhibitors including 4-aminopyridine, 7-methoxytacrine, amiridine, besipirdine, CHF2819, CI-1002, DMP 543, donepezil, eptastigmine, galantamine, huperzine A, huprine X, huprine Y, MDL 73745, metrifonate, P10358, P11012, phenserine, physostigmine, ouilostigmine, rivastigmine, Ro 46-5934, SM-10888, suronacrine, T-82, tacrine, TAK-147, tolserine, trifluoroacetophenone, TV3326, velnacrine, and zifrosilone; ACh release enhancers including linopirdine, and XE991; Choline uptake enhancers including MKC-231, and Z-4105; nicotinic agonists including: ABT-089, ABT-418, GTS-21, and SIB-1553A; NMDA antagonist including ketamine, and memantine; serotonin inhibitor such as cinanserin hydrochloride, fenclonine, fonazine mesylate, and xylamidine tosylate; serotonin antagonist including altanserin tartrate, aAmesergide, cyproheptadiene, granisetron, homochlorcyclizine, ketanserin, mescaline, mianserin, mirtazapine, periapine, pizotyline, olanzapine, ondansetron, oxetorone, risperidone, ritanserin, tropanserin hydrochloride, and zatosetron; serotonin agonists including 2-methylserotonin, 8-hydroxy-DPAT, buspirone, gepirone, ipsapirone, rizatriptan, sumatriptan, and zolmatriptan; serotonin reuptake inhibitors including citalopram, escitalopram oxalate, fluoxetine, fluvoxamine, paroxetine, and sertraline; dopamine antagonists including pimozide, ouetiapine, metoclopramide, and dopamine precursors including levodopa in diagonising of a neurological condition, wherein said compound is used as a probe compound.

According to still another aspect, the present invention relates to the use of the compound scopolamine for initiating a neurological reaction for dementia of the Alzheimer's type (AD group).

According to still another aspect, the present invention relates to the use of software to compare data measured on a control subject with data measured on a subject suspected to suffer from a neurological condition, wherein said software is able to perform the following steps

using received biosignal data obtained from biosignal measuring device for determining one or more features, said biosignal data being obtained after administering said at least one compound,
calculating posterior probability vector for said subject in accordance to posterior probability vectors obtained from reference subjects from at least one group, said posterior probability vectors consisting of probability values associated to feature or a feature combination elements determined from biosignal data for said reference subjects, said posterior probability vectors resulting in a statistical distribution of said features or feature combinations for said reference subjects,
comparing the posterior probability vector for said subject with a distribution

In still another embodiment, the present Invention relates to a method for assessing a neurological condition in a subject comprising:

administering to the subject a probe compound that has a neurophysiological effect, performing one or more biosignal measurements on the subject to obtain multidimensional biosignal data;
analyzing said multidimensional biosignal data with multidimensional analytical techniques to determine the presence of a discriminatory pattern which indicates that the subject is afflicted with or has a predisposition for said neurological condition.

In an embodiment, said one or more biosignal measurements on the subject which comprise an electroencephalographic measurement.

In an embodiment, said biosignal measurements are performed both prior to and after said administration of said probe compound.

In an embodiment, said the neurological condition is selected from the group consisting of Alzheimer's disease, multiple schlerosis, mental conditions including depressive disorders, bipolar disorder and schizophrenic disorders, Parkinsons' disease, epilepsy, migraine, Vascular Dementia (VaD), Fronto-temporal dementia, Lewy bodies dementia, Creutzfeld-Jacob disease and vCJD (“mad cow” disease).

In an embodiment, said one or more biosignal measurements comprise a biosignal measurement selected from the group consisting of magnetic resonance imaging (MRI), functional magnetic resonance imaging (FMRI), magnetoencephalographic (MEG) measurements, positron emission tomography (PET), CAT scanning (Computed Axial Tomography) and single photon emission computerized tomography (SPECT).

In an embodiment, said at least one probe compound is selected from the group of said compounds.

In an embodiment, said method further comprises subjecting the subject to a sensory stimulus prior to or during the electroencephalographic measurement.

In an embodiment, said discriminatory pattern is obtained with the above mentioned method.

The aspects of the present invention may each be combined with any of the other aspects. These and other aspects of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 illustrates schematically an interaction between nerve cells occurs at the synapses,

FIG. 2 shows a method according to the present invention of generating a discriminatory signal for a neurological condition,

FIGS. 3-5 illustrate schematically a possible distribution for these properties for the reference subjects in groups A and B,

FIG. 6 shows the resulting feature extraction effect of selecting only those posterior probability vectors that have large variance for group A and B subjects,

FIG. 7 shows an example of the distribution for one property,

FIG. 8 illustrates the posterior probabilities for two subjects, an Alzheimer's subject, circles, and a control subject, crosses,

FIG. 9 illustrates the distribution for the Alzheimer's group and the control group in terms of the pca-posterior probabilities,

FIG. 10 illustrates an example of the recording protocol used in the method of the invention. In the figure number (1) represents time period when the subject is prepared for the test. Number (2) represents a two minute recording period at which the subject was instructed to be at rest. Number (39 represents the time period at which the probe compound is administered. Finally number (4) represents the two minute recording time period after administering the probe compound at which the subject was instructed to be at rest.

FIG. 11 is a block diagram of the data acquisition system. In the figure number (8) represents the test subject. EEG data acquisition system is represented in (9) including an amplifier (11) and analog digital converter (12). The digitized data is next passed onto the computing system (13), including CPU (6), programmable memory (5) and data store (10). The operator can view in real time the data acquisition process on the monitor (7),

FIG. 12 shows a block diagram of the classification ensemble,

FIGS. 13 and 14 illustrate the effect of the scopolamine,

FIG. 15 illustrates the comparison between the classification performances for the two sets evaluated using the 3-NN scheme, and

FIG. 16 illustrates the same comparison, but using the SVM classification scheme to obtain the feature pair classifiers.

### DESCRIPTION OF EMBODIMENTS

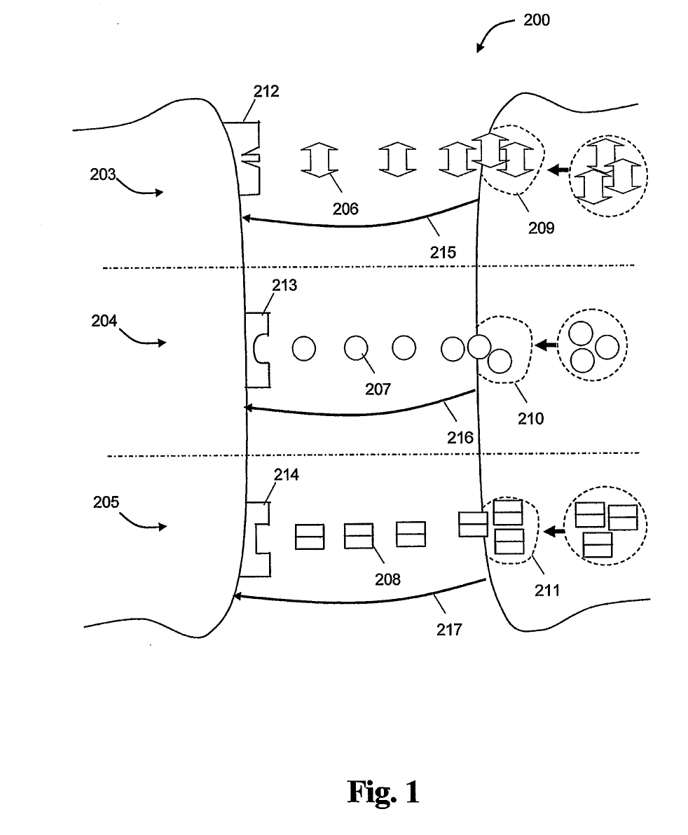

A neurophysiologic condition depends on interaction between different nerve cells. Referring to FIG. 1 interaction between nerve cells occurs at the synapses 200, e.g. between an axon 201 of one cell and a dendrite 202 of another cell. The interaction is through a multiple of neurotransmitter systems 203, 204, 205. Each neurotransmitter system has distinct neurotransmitters 206, 207, 208 that are released from vesicles 209, 210, 211 in the axon upon interaction which are in turn received by receptors 212, 213, 214, on the dendrite, that are distinct for each neurotransmitter system.

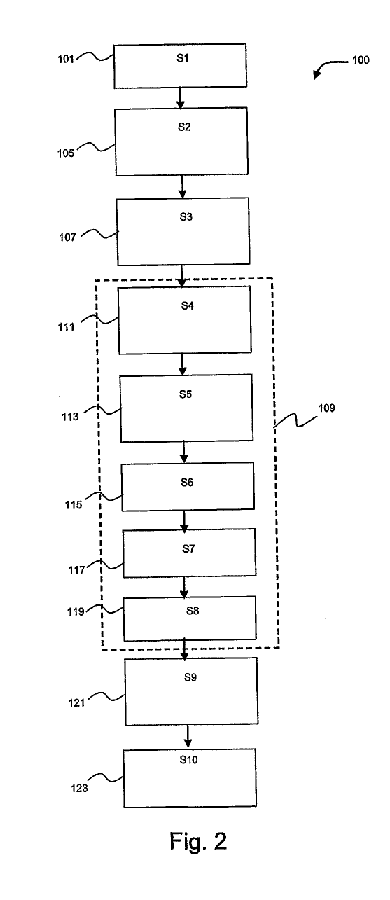

Referring to FIG. 2 a method 100 of generating a discriminatory signal for a neurological condition is shown.

At least one probe compound that has a neurophysiologic effect is provided (S1) 101. This probe compound is adapted to initiate a neurological reaction when administered to a subject, wherein the selection of the compound must be such that the neurological reaction caused for a subject suffering from a particular neurological disease, here below referred to as a patient, is different than that caused for a healthy subject, here below referred to as a reference person. Accordingly, administering the compound to a reference subject, e.g. a healthy subject, and subject suffering from a neurological disease causes a divergence in the neurological reaction between the two subjects that separates the two subjects. In more technical terms, the term ‘probe compound’ is used in this context to indicate a compound with a neurophysiologic effect and which perturbs a biophysical pathway/signal which can be related to the neurological condition in question, i.e. a probe compound is selected which affects differently a subject suffering from said condition and an individual not afflicted with the condition.

However, this difference may or may not be readily apparent or known a priori, thus the one or more probe compounds may be selected from compounds with a known neurophysiologic effect, and the method of the invention will recognize possible different effects on individuals with a particular condition and control individuals not afflicted with said condition, i.e. Identify useful probe compounds. Among potentially useful probe compounds are compounds from the group of consisting of GABA affecting drugs including: propofol and etomidate; barbiturates including methohexital, thiopental, thiamylal, buthalital, thialbarbital, hexobarbital, pentobarbital, secobarbital, hexethal, butalbital, cyclobarbital, talbutal, phenobarbital, mephobarbital, and barbital;

benzodiazepines such as: alprazolam, bromazepam, chlordiazepoxide, clobazam, clonazepam, clorazepate, clozapine, olanazapine diazepam, estazolam, flunitrazepam, flurazepam, halazepam, ketazolam, loprazolam, lorazepam, lormetazepam, medazepam, midazolam, nitrazepam, nordazepam, oxazepam, prazepam, ouazepam, temazepam, and triazolam;
cholinergic agonists such as aceclidine, AF-30, AF150, AF267B, alvameline, arecoline, bethanechol, CDD-0102, CDD-0034-C, CDD-0097-A, cevimeline, CI 1017, cis-dioxolane, milameline, muscarine, oxotremorine, pilocarpine, RS86, RU 35963, RU 47213, sabcomeline, SDZ-210-086, SR 46559A, talsaclidine, tazomeline, UH5, xanomeline, and YM 796;
cholinergic antagonists including: AF-DX 116, anisotropine, aprophen, AQ-RA 741, atropin, belladonna, benactyzine, benztropine, BIBN 99, DIBD, cisapride, clidinium, darifenacin, dicyclomine, glycopyrrolate, homatropine, atropine, hyoscyamine, ipratropium, mepenzolate, methantheline, methscopolamine, PG-9, pirenzepine, propantheline, SCH-57790; SCH-72788, SCH-217443, scopolamine, tiotropium, tolterodine, and trihexyphenidyl;
acetyl choline esterase (ACE) inhibitors Including: 4-aminopyridine, 7-methoxytacrine, amiridine, besipirdine, CHF2819, CI-1002, DMP 543, donepezil, eptastigmine, galantamine, huperzine A, huprine X, huprine Y, MDL 73745, metrifonate, P10358, P11012, phenserine, physostigmine, ouilostigmine, rivastigmine, Ro 46-5934, SM-10888, suronacrine, T-82, tacrine, TAK-147, tolserine, trifluoroacetophenone, TV3326, velnacrine, and zifrosilone;
ACh release enhancers including: linopirdine, and XE991;
Choline uptake enhancers including: MKC-231, and Z-4105;
nicotinic agonists including: ABT-089, ABT-418, GTS-21, and SIB-1553A;
NMDA antagonist including: ketamine, and memantine;
serotonin inhibitor such as: cinanserin hydrochloride, fenclonine, fonazine mesylate, and xylamidine tosylate;
serotonin antagonist including: altanserin tartrate, aAmesergide, cyproheptadiene, granisetron, homochlorcyclizine, ketanserin, mescaline, mianserin, mirtazapine, perlapine, pizotyline, olanzapine, ondansetron, oxetorone, risperidone, ritanserin, tropanserin hydrochloride, and zatosetron;
serotonin agonists including: 2-methylserotonin, 8-hydroxy-DPAT, buspirone, gepirone, ipsapirone, rizatriptan, sumatriptan, and zolmatriptan;
serotonin reuptake inhibitors including: citalopram, escitalopram oxalate, fluoxetine, fluvoxamine, paroxetine, and sertraline;
dopamine antagonists including pimozide, ouetiapine, metoclopramide, and dopamine precursors including: levodopa,
as well as other compounds that affect the brain and nerve system.

It is important to realize that a certain neurological condition, e.g. a particular disease, is rarely if ever related to changes in a single biophysical pathway, e.g. a distinct neurotransmitter system, but rather the condition affects a range of pathways. However, in certain cases it known that symptoms related to certain neurological condition are due to changes in particular systems, e.g. decreased short term memory in Alzheimer's disease has been related to the cholinergic system, attention deficiency in children with attention deficit hyperactive disorder (ADHD) and attention deficit disorder (ADD) has been related to the dopamine system.

As illustrated in the accompanying example, the method typically relies on at least one group of diseased individuals, i.e., individuals pre-diagnosed with a particular condition of interest, and at least one group of control individuals who are not afflicted with the condition being assessed. The size of the groups is selected to give statistically sound data.

All individuals that are measured are administered the same dose of the one or more probe compounds and preferably the time interval between the administration and the onset of post-administration measurements is substantially identical for all measured individuals.

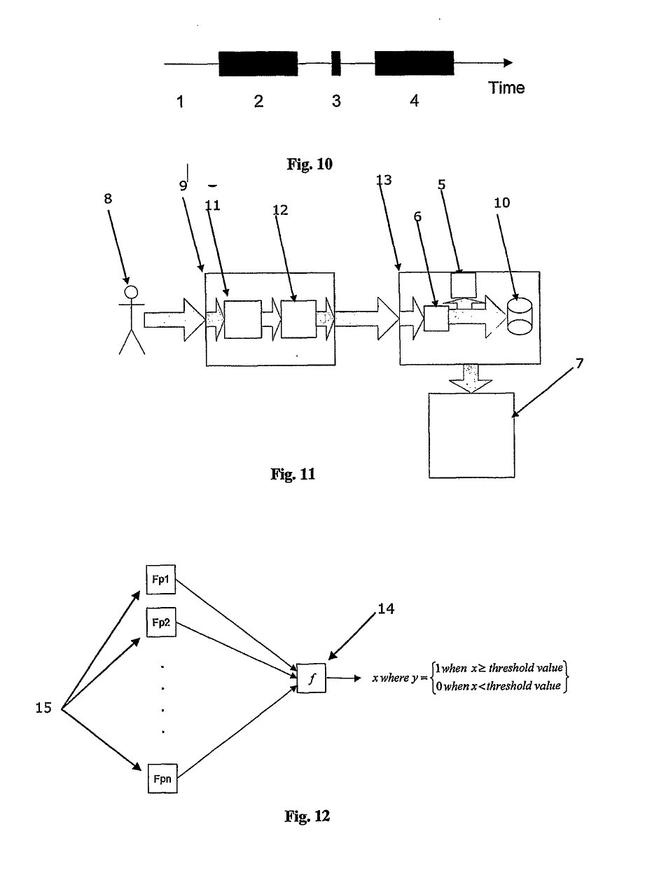

In preferred embodiments, as illustrated schematically in FIG. 10, biosignal measurements are obtained both before 2 as well as after 4 the probe compound administration 3. In this way analysis of the combined data in accordance with the invention can more effectively pick up differential effects of the probe compound and thus generate a more decisive discriminatory signal for indicating presence of the condition being studied.

It is particularly appreciated that the present invention is useful for generating discriminatory signals for assessing and/or diagnosing various conditions with a neurological aspect, which are not readily accurately diagnosed based on their symptoms and complex and varying physiological effects. Such conditions and diseases include without limitation Alzheimer's disease, multiple schlerosis, mental conditions including depressive disorders, bipolar disorder and schizophrenic disorders, Parkinsons' disease, epilepsy, migraine, Vascular Dementia (VaD), Fronto-temperal dementia, Lewy bodies dementia, Creutzfeld-Jacob disease, attention deficit disorder (ADD), attention deficit hyperactive disorder (ADHD), anxiety disorder, conduct disorder, oppositional defiant disorder, Tourette syndrome and vCJD (“mad cow” disease).

In this embodiment, biosignal data are obtained from the subject suffering from a neurological condition, i.e. a patient, both prior (S2) 105 and subsequent (posterior) (S3) 107 to administering the at least one probe compound. One or more biosignal measurements comprise biosignal data selected e.g. from the group consisting of electroencephalography (EEG) magnetic resonance imaging (MRI), functional magnetic resonance imaging (FMRI), magneto encephalography (MEG) measurements, positron emission tomography (PET), CAT scanning (Computed Axial Tomography) and single photon emission computerized tomography (SPECT). The biosignal measurement can also be related to other physiological parameters, e.g. gene expression levels in blood or other tissues found e.g. by micro-arrays or pcr, concentration of specific proteins and enzymes in e.g. blood and urine samples, basic physiological parameters such as temperature, sex, age, ethnic origin, weight, height and so forth. Furthermore the biosignal data may be of environmental or historic origin, e.g. main occupation, disease history, living conditions, diet, details of drug and alcohol use, smoking and so forth. In all cases it is implied that the data is computerized, e.g. images digitized and so forth.

The biosignal data obtained prior (S2) 105 to administering the probe compound may therefore be used as e.g. background data for the data (S3) 107 taken subsequent to administering the probe compound. The additional neurological effect obtained by administering the probe compound may therefore be more effective, e.g. by subtracting the biosignal data obtained prior (52) 105 to administering the probe, than in the absence of the background data. Also, the biosignal data obtained prior (S2) 105 to administering the probe compound may in some situations provide additional information when calculating feature values, which will be described in more details later. Steps (S2) 105 and (S3) 107 can be repeated for several compounds, since each compound may challenge different physiological pathways. In that way data is obtained that reflects the state of multiple neurotransmitter systems.

In an embodiment it may be sufficient utilize only data obtained posterior (S3) 107 to administration of the probe compound.

The subsequent step of the method shown in FIG. 2 relates to defining reference values 109 from a group or groups of reference persons. This step relates more to generating feature values, that will be discussed in more details here below, to be used as a reference values for determining whether the subject belongs to a particular group or not. This step relates to e.g. constructing a database of the reference values to be used for distinguishing whether the patient is diseased or not.

The selection of the group of the reference persons is typically based on the characteristics of the group.

As shown here, step 109 is divided into five sub-steps (S4) 111-(S8) 119 starting with obtaining biosignal data from the reference persons both prior (S4) 111 and subsequent (S5) 113 to administering the amount of the same probe compound. Here, it is of course preferred or even essential that the administered probe compound and the amount of it administered to each of the reference persons and the patient is precisely the same. The dose is selected such that the expected response would be saturated. The reference subjects are preferably classified according to existing medical records and thorough examination by medical doctors who determine whether each subject belongs to the target groups, e.g. groups with particular neurological conditions such as dementia of the Alzheimer's type. In one embodiment such biosignal data is collected during controlled clinical trials where each subject undergoes thorough medical examination by specialist medical doctors, or other types of skilled person e.g. skilled technicians, who determine whether trial candidates fulfill the prescribed definition for each group.

Based on the data obtaining from the reference group, one or more reference features are defined (S6) 115. This is performed by “pre-scanning” the data from each of the reference persons and check which features are suitable to be used as reference features. A basic condition that needs to be fulfilled when defining the reference features is that there is a correlation between the features in the data obtained subsequent, or both prior and subsequent, to administering the compound for preferably all the test persons. An example of such reference features when the biosignal originates from EEG e.g. the absolute delta power, absolute theta power, relative theta power, spectral frequency, total power, DFA scaling exponent (alpha band oscillations) etc. More exhaustive list of such reference features is listed later in the description. Accordingly, based on the type of disease and the associated type of compound that is preferred to use, some reference features may be more suitable than other reference features. As an example, for a disease A and the compound A′ to be administered, it might be preferred to use the absolute delta power, absolute theta power, relative theta power due to the high correlation in these features between the test persons, whereas for a disease B and the compound B′ to be administered it might be preferred to use relative theta power, spectral frequency, total power, DFA scaling exponent (alpha band oscillations) as features. The reference features can also include relations between the features in both the data obtained prior to the administering the compound (S4) 111 and subsequent to the administering the compound (S5) 113. As an example of such relation is the absolute delta power from the data subsequent to administering the compound divided with the absolute delta power from the data prior to administering the compound. Accordingly, one reference feature can give 2 or more different reference features, e.g. delta power (after)/delta power (prior); delta power (prior)/delta power (after), delta power (prior)*delta power (after) etc.

Subsequently, the feature values that are associated to the defined features are calculated (S7) 117. In the above mentioned examples, this corresponds to calculating the delta power, absolute theta power, relative theta power values, or power, spectral frequency, total power, DFA scaling exponent (alpha band oscillations) values. These calculated values are then used to determine posterior probability vectors (S8) 119 for each respective reference subject within one or more reference groups, wherein the posterior probability vectors result in a distribution of said features or feature combinations for said reference subjects. The resulting distribution could e.g. be a Gaussian distribution. This will be clarified here below and followed by an example.

In one embodiment all the features are taken into account, where the weight the features should carry is determined in order to maximally separate the groups under consideration. Let fᵢ, iε{1, 2, . . . , N_{f}} denote the set of features calculated from the bio-signal data, and N_{f} is the number of features considered. Consider two balanced groups A and B, in the sense that the number of subjects in each group is roughly equal. Let N_{S} denote the total number of subjects in the groups. In praxis it is not feasible to consider all the features at once while solving a classification problem the main danger being over-fitting the data. Let N denote the number of features considered at once. A rule of thumb is not to consider more than N≦^{N}^{_{S}}/₁₀. Let Vᵢε(fᵢ_{₁}, fᵢ_{₂}, . . . , fᵢ_{_{N}}) be the set of all non-repetitive combinations of features, these combinations are termed properties. As an example consider f={1, 2, 3} and N=2 then V={(1,2),(1,3),(2,3)}. For each element i In V a classifier is found. The classifier is then used to estimate the posterior probabilities for each subject with respect to one of groups, e.g. the probability that subject j belongs to the group A, estimated from the distribution of the features in Vᵢ. The classifier is a multidimensional pattern recognition method e.g. k-NN nearest neighbor scheme (k-NN), support vector machines (SVM), linear discriminant analysis, quadratic discriminant analysis, regularized discriminant analysis, Logistic regression, naive Bayes classifier, hidden Markov model classifiers, neural network based classifiers including multi-layer perception networks and radial basis function networks, support vector machines (SVM), Least-squares SVM, Classification Tree-based classifiers including Classification and Regression Trees (CART), ID3, C4.5, C5.0, AdaBoost, and ArcX4, Tree-based ensemble classifier including Random Forests™. The chosen classifier is then used to construct a boundary in feature space that best separates the groups according to criteria the particular classifier is based on. From the found boundary the posterior probability is estimated, usually as function of distance from the boundary and from the estimated distribution of the training set, e.g. the data from the groups, parameterized as a function of the distance from the said boundary. These probabilities are denoted Pⱼᵢ. Consider an ideal property k in the sense that P_{fk}=1 for all subjects j belonging to group A and Pⱼₖ=0 for all subjects j belonging to group B. That would indicate that the classifier classifies correctly all subjects in the training set and, provided that over fitting has not occurred, that it is a good predictor. It turns out that such an ideal distribution of Pⱼᵢ for a particular property is a distribution that maximizes the variance of Pⱼᵢ. Principal component analysis (PCA) Identifies independent combinations of variables, i.e. uncorrelated, which maximize the variance. In praxis PCA is performed by examining the eigenvectors and corresponding eigenvalues of the normalized correlation matrix. These eigenvectors are termed pca-eigenvectors. The pca-eigenvector with the largest eigenvalue is the independent combination of properties that maximizes the variance; the pca-eigenvector with the second largest eigenvalue describes the combination with second largest variance and so forth. In other words, the variance of the combinations described by the pca-eigenvectors of the normalized correlation matrix is proportional to their eigenvalues. Let W be the matrix constructed by the pca-eigenvectors, i.e. the vectors form the columns of W. The pca-posterior probability matrix is defined by P^{pca}=PW. After this mapping, columns corresponding to pca-eigenvectors with maximum eigenvalues will be as close as possible to the ideal posterior probability distribution with respect to classification. We can consider several such columns and repeat the classification in pca-posterior probability space. In this embodiment the reference feature values calculated (S7) 117 are based on all the actual chosen features and are in fact the pca-posterior probabilities. In a worked database one could store the raw data, the features of the reference groups in order to build the property maps, the pca-posterior probability values for all properties, the matrix W and the corresponding eigenvalues.

When a new subject is to be classified, e.g. a potential patient to be diagnosed, features are calculated (S9) 121 from the biosignal data obtained in steps (52) 105 and (S3) 107. The results are subsequently compared to the data in the database that contains the reference feature values (S10) 123. The procedure is then to determine the posterior probabilities for the new subject in the same manner as in step 109 described above for each property resulting in the posterior probability vector for the subject, P^{subj}=(P₁^{subj}, P₂^{subj}, . . . ). Then the stored matrix W is used to find the corresponding pca-posterior probabilities, P^{pca,subj}=P^{subj}W. Choosing the most relevant components according to the eigenvalues of the matrix W a new classification is performed with respect to the same components in P^{pca} obtained from the database. Here only one multi dimensional classification is performed using the chosen components of P^{pca} and comparing them to the training data that consists of the same chosen components from P^{pca}. This will for example result in a single posterior probability that the new subject belongs to a particular group and is the basis for classification, e.g. prediction that said subject belongs to the particular group. In praxis decision, classification or diagnosis is made based on a chosen cutoff probability that corresponds to an accepted confidence level. A full diagnosis can involve several such classifications where the subject is compared to several characteristic groups.

In an embodiment, the step of obtaining the data from both the patient comprises obtaining the data during similar activity as performed by the reference persons during obtaining the reference data. As an example, if the data form the reference persons where obtained while they had their eyes closed, it is preferred that the data obtained from the patient is obtained while he/she has the eyes closed, or if the data obtained from the reference persons where obtained while observing an image or a text, a similar activity should be performed by the patient during obtaining the data.

### Example 1

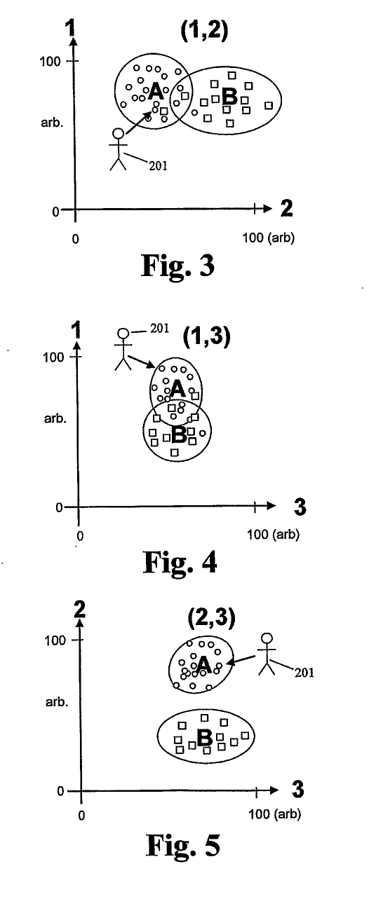

Assuming we have two groups of reference subjects, group A and group B where f={1, 2, 3} is the set of features that are used and N=2 is a combination parameter that determines the number of features to be combined (e.g. two features can be combined together or three features etc). The set of all non-repetitive combinations of features will be: V={(1,2),(1,3),(2,3)}, i.e. the first element is a combination of feature 1 and 2, the second is a combination of feature 1 and 3 etc. Based on the above, (1,2) is a first property, (1,3) is a second property and (2,3) is the third property. FIGS. 3-5 illustrate schematically a possible distribution for these properties for all the reference subjects in groups A and B. FIG. 3 shows the statistical distribution of the property (1,2) property for the two groups A and B where the reference subjects in the groups are plotted in accordance to the (“1”,“2”) feature values (i.e. “1” is the feature 1 value and “2” is the feature 2 value). The domain A shows the distribution of the reference subjects (marked with circles) in group A and domain B shows the distribution for the reference subjects in group B (marked with squares). FIGS. 4 and 5 show the corresponding statistical distributions of properties (1,3) and (2,3), respectively, i.e. for the (1,3) distribution of property as shown in FIG. 4 all the (“1”,“3”) feature values for all the reference subjects are plotted and for the (2,3) statistical distribution in FIG. 5 all the (“2”,“3”) feature values for all the reference subjects are plotted.

Continuing with the example, the subsequent step is to calculate the posterior probability vector for each respective person in groups A and B. For clarification, an example of feature values (“1”,“2”), (“1”,“3”) and (“2”,“3”) for subject 201 that is classified in group A are shown in FIGS. 3-5. The probability vector for this particular subject is determined by calculating the probability that the subject 201 belongs to group A. For this particular subject, it is clear that the subject lies within domain A and not at the boundaries or even within domain B which results in a posterior probability vector having elements of high probability for each respective property, i.e. the variance of the vector is high. The result of the posterior probability vector could accordingly be P=[0.79;0.85;1.0], i.e. the probability the subject 201 lies within domain A for properties (1,2), (1,3) and (2,3) is 0.79, 0.85 and 1.0, respectively. As mentioned previously, the posterior probability vector is calculated for all the subjects in domains A and B. For the subjects in group B, the resulting posterior probability vectors will have low values since group B is being used as a reference group. Accordingly, a posterior probability vector P=[0.09;0.05;0.0] for a subject in group B indicates that the probability that the subject B is within group B is (very) large, i.e. the variance is large.

Furthermore, after the calculation an evaluation process is performed for evaluating which posterior probability vectors are to be used i.e. what posterior probability vectors have sufficient large variance. This “filtering process” is essential for performing a feature extraction. As an example, instead of 0.79, 0.85 and 1.0 values, the result for another subject from group A could be 0.5, 0.49 and 1.0. (this could be a person that lies within the overlapped domain). In this case the posterior probability vector might not be used due to the low variance. For evaluating the posterior probability vectors a threshold value may be defined whereby all posterior probability vectors having variance below the pre-defined threshold value may not to be used. As an example, for a threshold value=0.6 for the subjects in group A it might be sufficient that only one element in the posterior probability vector is below 0.6 for not using the probability vector, or only that specific element might not be used.

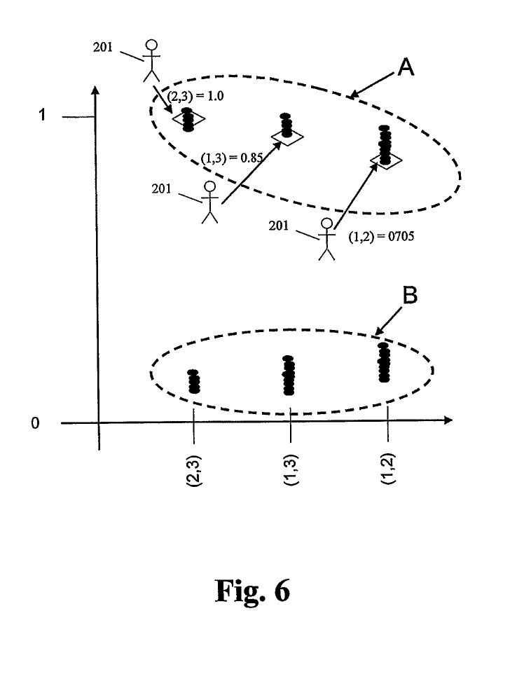

FIG. 6 shows the resulting feature extraction effect of selecting only those probability vectors that have large variance for group A and B subjects since such a “filtering process” selects only those posterior probability vectors having large variance. In this graph the selected posterior probability vectors are plotted for all the reference subjects, where the x-axis stands for the property elements (1,2), (1,3), (2,3), and the y-axis for the associated probability values. For clarification, the posterior probability vector P=[0.79;0.85;1.0] for subject 201 from group A is shown for the three property elements, along with other posterior probability vectors from other reference subjects form groups A and B (marked with filled circles). Below in FIG. 6 is the result for the subjects from group B. It should be inherent from the example that since group B was selected as a reference group when calculating the posterior probability vectors, all the reference subject from group A lie in the upper part and the reference subjects B lie in the lower part.

### Example 2

The effectiveness of the invention has been verified in a clinical trial. The participants in the trial were divided in two groups. One group consisted of 10 elderly subjects that have been diagnosed with, mild to moderate, dementia of the Alzheimer's type (AD group). A second age-matched group of 10 healthy individuals (i.e. non-AD individuals) was included as a control group.

The AD-group of participants consisted of patients in follow-up surveillance in the memory clinic at the Department of Geriatrics in Landspitali University Hospital, Reykjavik, Iceland. The group consisted of patients with Alzheimer's Disease (AD) (N=10) according to ICD-10. The other group consisted of normal Control participants (N=10), who were recruited from relatives of demented patients attending a day-care center.

To be eligible for participation in the study the subjects had to be in the range of 60-80 years of age, in good general health as determined by standard physical examination, with no acute changes on ECG. Exclusion criteria included smoking or any other use of tobacco (also excluding those that had stopped tobacco use about a week or less prior to the trial), treatment with neuroleptics and benzodiazepines, impaired liver- or kidney function, hypersensitivity to scopolamine, indication of drug, alcohol or medical abuse, glaucoma or possibility of raised intraocular pressure with administration of scopolamine. Prior to the screening visit the subjects were interviewed by phone. The AD subjects were selected from hospital records. All the AD patients in the follow-up program at the Memory Clinic were being treated with anti-dementia drugs. To minimize the variation between the subjects in the trial, the participants in the AD-group were selected from patients that were being treated with the same cholinesterase-inhibitor, Reminyl® (galantamine HBr).

In the screening visit the participants underwent physical examination by the study physician and fulfillment of the inclusion/exclusion criteria set fourth. Information of diagnosis, ECG recording, blood sampling, staging (Global Deterioration Scale (GDS) and MMSE (see Table 1), and CT/SPECT were recorded and finally an examination was carried out by an ophthalmologist.

Electroencephalographic neurophysiological signals were recorded from each of the subjects. The recording protocol was divided into two parts 105, 107 or sessions which were identical. In between the sessions the provided substance 101 substance scopolamine was administered intravenously, see FIG. 10. Within each section a two-minute period was recorded while the subject was instructed to be at rest with eyes closed. The data collected from these periods were used to estimate the individual features. The substance scopolamine was chosen based on its effects perturbing biophysical pathways that are known to deteriorate in subjects suffering from Alzheimer's disease. Scopolamine is a cholinergic antagonist, and it is well known that the cholinergic system deteriorates in Alzheimer's disease patients.

TABLE 1
Characteristics of the participants examined in the study.

| | Number | Male | Female | Average Age | GDS# | MMSE |
|---|---|---|---|---|---|---|
| Controls | 10 | 3 | 7 | 72.6 | 1.2 | 29.1/30 |
| | | | | SD 5.3 | SD 0.4 | SD 0.9 |
| AD | 10 | 7 | 3 | 75.9 | 4.3 | 21.2/30 |
| | | | | SD 3.0 | SD 0.5 | SD 2.6 |

#Global Deterioration Scale (GDS) for age-associated cognitive decline and Alzheimer's disease: stage 1: No cognitive decline (normal), stage 2: very mild cognetive decline (forgetfulness); stage 3: mild cognitive decline (early confusional); stage 4: moderate cognitive decline (late confusional); stage 5: Moderately severe decline (early dementia); Stage 6: severe cognitive decline and stage 7: very severe cognitive decline. The last two did not participate in this study. SD indicates one standard deviation from the mean.

The electroencephalographic signals were recorded using computerized measuring equipment, see FIG. 11. The recordings were performed using the conventional International 10-20 system of electrode placement. The collected data is stored in raw format on a storage device for later analysis. During the recordings the signals are displayed simultaneously on a computer screen 7. This allows the operator to monitor if electrodes come loose and to enter marks that indicate specific events. Such events may indicate initiation of specific parts of the recording protocol or occurrences that may lead to artifacts being present in the recordings. Such occurrences include that the subject blinks his eyes, swallows, moves or in general breaches protocol.

When all the data had been collected features that characterize the individual recordings were extracted. Identical features were extracted from the first and second recording sessions of the protocol. Features extracted were derived from results reported in the scientific literature (Adler G. et al. 2003, Babiloni C. et al. 2004, Bennys K. et al. 2001, Brunovsky M. et al. 2003, Cichocki et al. 2004, Cho S. Y. 2003, Claus J. J. et al. 1999, Hara J. et al. 1999, Holschnelder D. P. et al. 2000, Hongzhi Q. I. et al. 2004, Huang C. et al. 2000, Hyung-Rae K. et al. 1999, Jelles B. et al. 1999, Jeong J. et al. 1998, 2001, 2004, Jonkman E. J. 1997, Kikuchi M. et al. 2002, Koenig T. et al. 2004, Locatelli T. et al. 1998, Londos E. et al. 2003, Montplaisir J. et al. 1998, Moretti et al. 2004, Musha T. et al. 2002, Pijnenburg Y. A. L. et al. 2004, Pucci E. et al. 1998, 1999, Rodriquez G. et al. 1999, Signorino M. et al. 1995, Stam C. J. et al. 2003, 2004, Stevens A. et al. 1998, 2001, Strik W. K. et al. 1997, Vesna J. et al. 2000, Wada Y. et al. 1998, Benvenuto J. et al. 2002, Jimenez-Escrig A. et al. 2001, Sumi N. et al. 2000).

The features used in the example were numbered as follows. 16 base features were selected.

1. Absolute delta power
2. Absolute theta power
3. Absolute alpha power
4. Absolute beta power
5. Absolute gamma power
6. Relative delta power
7. Relative theta power
8. Relative alpha power
9. Relative beta power
10. Relative gamma power
11. Total power
12. Peak frequency
13. Median frequency
14. Spectral entropy
15. DFA scaling exponent (alpha band oscillations)
16. DFA scaling exponent (beta band oscillations).

These features were evaluated using a part of the first section where the subjects were at rest with eyes closed. The same features were estimated for the corresponding second section which occurs after administration of the scopolamine. These features post-administration are enumerated 17-32. Finally the features are combined in order to obtain a measure of the response of each feature to the administration of the scopolamine, by determining the ratio of the same feature before and after drug administration. These combination features are enumerated 33-48. (For example feature 33 is the ratio of feature 1 and 17.) Many other combinations of the features before and after administration will reflect the response as well, e.g. the difference.

In order to demonstrate the effectiveness of using a probe compound 101 the following analysis was performed. The features were used to classify the two groups using a pattern classification scheme.

In order to design a classifier a labeled training set is required (supervised learning). The classifier is then used to classify unseen data. In order to evaluate the performance of a classifier an independent test set is required.

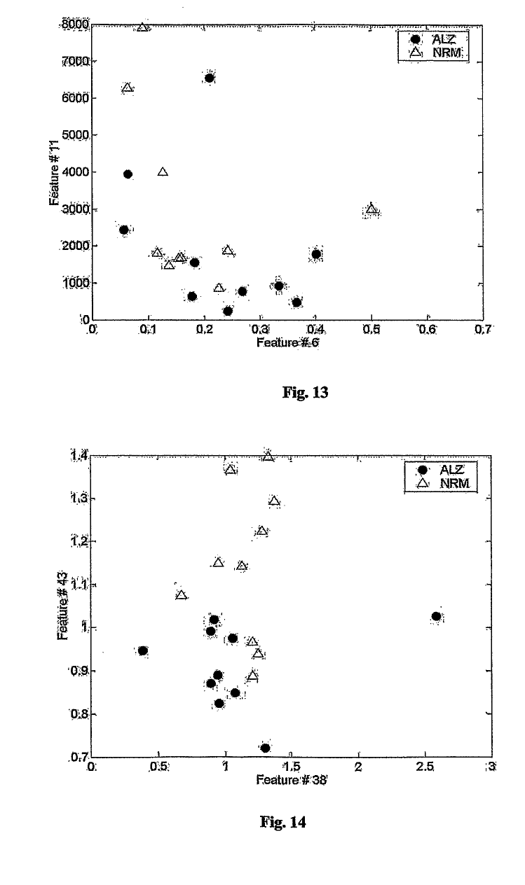

A training dataset with two groups (10 in each) cannot support classification taking into account more than 2 features at the time without risk of running into over fitting problems. Over fitting leads to classifiers that in general perform poorly on unseen data. In the present example two features at a time were considered. FIGS. 13 and 14 illustrate the effect of the scopolamine. In FIG. 13 the features stem from measurements before administration of the scopolamine, while FIG. 14 demonstrates the response of the same features by considering the ratio of their values pre- and post-administration. Evidently the scopolamine leads to significantly better separation between the groups for this feature pair.

All possible combinations of features were considered. Hence, if d features are taken into account d(d+1)/2 possible pairs were tested. For each pair the classification performance or accuracy was estimated by applying the “leave one out” scheme as follows. Let N be the total number of elements in the training set. The scheme is based on constructing N new training sets each with N−1 elements, where each element of the initial training set is left out once. For each resulting training set the element left out constitutes the test set. The overall performance is estimated with ratio of incorrect classifications of test sets to N.

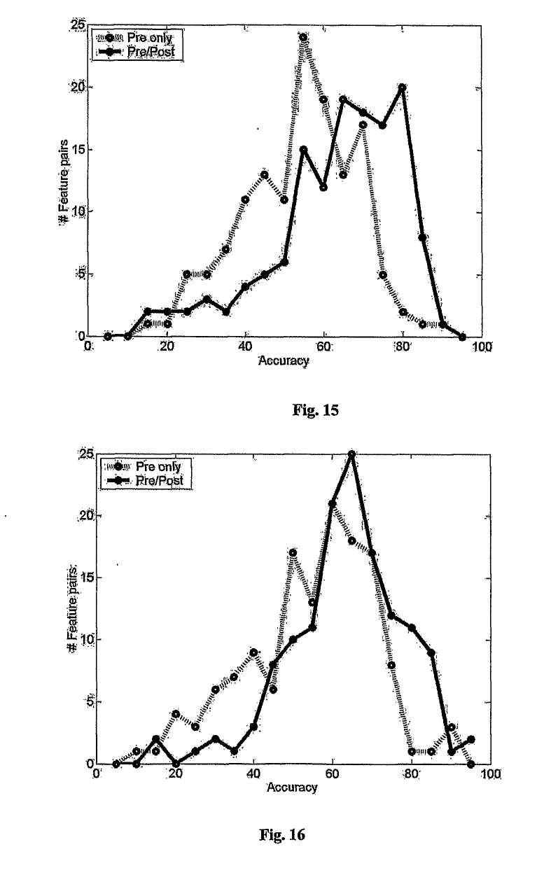

The efficiency of applying a pattern enhancing substance, scopolamine in this example, was demonstrated by considering the histogram of classification performance for two distinct feature sets, the set that only involves features extracted prior to substance administration, features 1-16, and the set that is sensitive to the response to the pattern enhancing substance, features 33-48, i.e., the ratios of the basic features before and after administration. The sets are of equal size. The features were estimated from the P3-P4 montage. FIG. 15 illustrates the comparison between the classification performances for the two sets evaluated using the 3-NN scheme. As is evident for this example, the pattern enhancing substance leads to substantially enhanced classification performance. The number of feature pair classifiers scoring 80% or better goes from 4 to 29. FIG. 16 illustrates the same comparison, but using the SVM classification scheme to obtain the feature pair classifiers. The number of feature pair classifiers scoring 80% or better goes from 5 to 23. This demonstrates that using a probe compound leads to a signal that is more discriminatory.

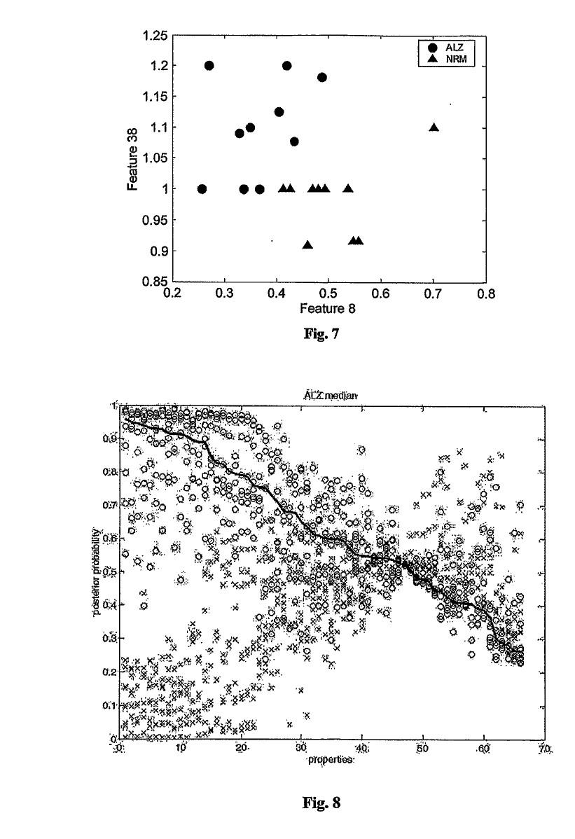

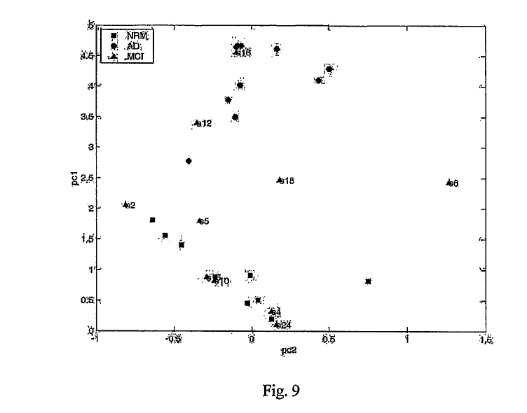

Next we demonstrate how the database is constructed. Again working with two features in each property, FIG. 7 shows the distribution for one property. For each property the posterior probability is calculated using a SVM classifier. FIG. 8 illustrates the posterior probabilities for two subjects, an Alzheimer's subject, circles, and a control subject, crosses. The solid line illustrates the median for the whole Alzheimer's group. It is practical in order to minimize destructive interference to include only properties with median posterior probability above some chosen threshold, e.g. 0.8. FIG. 9 illustrates the distribution for the Alzheimer's group and the control group in terms of the pca-posterior probabilities.

In order to demonstrate the predictability, e.g. the diagnostic value, of the invention a third group was included in the clinical trial described above. This group was recruited from subjects that have been classified as having mild cognitive impairment (MCI). The group was age matched to the other groups. It is well known that about 12% of MCI subjects will receive diagnosis as Alzheimer's patients within one year. In FIG. 9 the results for this group are illustrated following the classification procedure outlined above, e.g. each subject is compared to the database constructed from the data from the first two groups. The invention predicted that subject s12 and s16 belong to the Alzheimer's group. The group was followed up one year after the clinical trial was conducted. It turned out that two subjects were diagnosed with dementia of the Alzheimer's type, the same subjects that the invention predicted belonged to that group one year prior to the follow up visit. This demonstrates that the invention is capable of detecting individuals with neurological condition dementia of the Alzheimer's type one year earlier than a conventional diagnosis workup is capable of. In other words the invention is capable of early diagnosis of Alzheimer's disease. Subjects 18 and s6 were predicted to belong to neither the Alzheimer's group nor the control group. Two years later these subjects were followed up for a standard workup, one was then known to have suffered a stroke while the condition of the other was uncertain. It was however clear that cognitive impairment was beyond doubt. It is speculated that these subjects have the vascular dementia or micro vascular dementia and should therefore not be classified with either group in the database in accordance with the result obtained from the invention.

The method in accordance with the invention for generating a discriminatory signal for a neurological condition comprises as mentioned above the step of administering to a subject suffering from said condition at least one probe compound that has a neurophysiologic effect.

As mentioned previously, the term ‘probe compound’ is used in this context to indicate a compound with a neurophysiologic effect and which perturbs a biophysical pathway/signal which can be related to the neurological condition in question, i.e. a probe compound is selected which affects differently a subject suffering from said condition and an individual not afflicted with the condition.

However, this difference may or may not be readily apparent or known a priori, thus the one or more probe compounds may be selected from compounds with a known neurophysiologic effect, and the method of the invention will recognize possible different effects on individuals with a particular condition and control individuals not afflicted with said condition, i.e. identify useful probe compounds. Among potentially useful probe compounds are compounds from the group of consisting of GABA affecting drugs including propofol and etomidate; barbiturates including methohexital, thiopental, thiamylal, buthalital, thialbarbital, hexobarbital, pentobarbital, secobarbital, hexethal, butalbital, cyclobarbital, talbutal, phenobarbital, mephobarbital, and barbital; benzodiazepines such as alprazolam, bromazepam, chlordiazepoxide, clobazam, clonazepam, clorazepate, clozapine, olanazapine diazepam, estazolam, flunitrazepam, flurazepam, halazepam, ketazolam, loprazolam, lorazepam, lormetazepam, medazepam, midazolam, nitrazepam, nordazepam, oxazepam, prazepam, ouazepam, temazepam, and triazolam; cholinergic agonists such as aceclidine, AF-30, AF150, AF267B, alvameline, arecoline, bethanechol, CDD-0102, CDD-0034-C, CDD-0097-A, cevimeline, CI 1017, cis-dioxolane, milameline, muscarine, oxotremorine, pilocarpine, RS86, RU 35963, RU 47213, sabcomeline, SDZ-210-086, SR 46559A, talsaclidine, tazomeline, UH5, xanomeline, and YM 796; cholinergic antagonists including AF-DX 116, anisotropine, aprophen, AQ-RA 741, atropin, belladonna, benactyzine, benztropine, BIBN 99, DIBD, cisapride, clidinium, darifenacin, dicyclomine, glycopyrrolate, homatropine, atropine, hyoscyamine, ipratroplum, mepenzolate, methantheline, methscopolamine, PG-9, pirenzepine, propantheline, SCH-57790; SCH-72788, SCH-217443, scopolamine, tiotropium, tolterodine, and trihexyphenidyl; acetyl choline esterase (ACE) inhibitors including 4-aminopyridine, 7-methoxytacrine, amiridine, besipirdine, CHF2819, CI-1002, DMP 543, donepezil, eptastigmine, galantamine, huperzine A, huprine X, huprine Y, MDL 73745, metrifonate, P10358, P11012, phenserine, physostigmine, oullostigmine, rivastigmine, Ro 46-5934, SM-10888, suronacrine, T-82, tacrine, TAK-147, tolserine, trifluoroacetophenone, TV3326, velnacrine, and zifrosilone; ACh release enhancers including linopirdine, and XE991; Choline uptake enhancers including MKC-231, and Z-4105; nicotinic agonists including: ABT-089, ABT-418, GTS-21, and SIB-1553A; NMDA antagonist including ketamine, and memantine; serotonin inhibitor such as cinanserin hydrochloride, fenclonine, fonazine mesylate, and xylamidine tosylate; serotonin antagonist including altanserin tartrate, aAmesergide, cyproheptadiene, granisetron, homochlorcyclizine, ketanserin, mescaline, mianserin, mirtazapine, perlapine, pizotyline, olanzapine, ondansetron, oxetorone, risperidone, ritanserin, tropanserin hydrochloride, and zatosetron; serotonin agonists including 2-methylserotonin, 8-hydroxy-DPAT, buspirone, gepirone, ipsapirone, rizatriptan, sumatriptan, and zolmatriptan; serotonin reuptake inhibitors including citalopram, escitalopram oxalate, fluoxetine, fluvoxamine, paroxetine, and sertraline; dopamine antagonists including pimozide, ouetiapine, metoclopramide, and dopamine precursors including levodopa, as well as other compounds that affect the brain and nerve system.

As illustrated in the accompanying example, the method typically relies on at least one group of diseased individuals, i.e., individuals pre-diagnosed with a particular condition of interest, and at least one group of control individuals who are not afflicted with the condition being assessed. The size of the groups is selected to give statistically sound data.

All individuals that are measured are administered the same dose of the one or more probe compounds and preferably the time interval between the administration and the onset of post-administration measurements is substantially identical for all measured individuals.

In preferred embodiments, as illustrated schematically in FIG. 10, biosignal measurements are obtained both before (2) as well as after (4) the probe compound administration (3). In this way analysis of the combined data in accordance with the invention can more effectively pick up differential effects of the probe compound and thus generate a more decisive discriminatory signal for indicating presence of the condition being studied.

It is particularly appreciated that the present invention is useful for generating discriminatory signals for assessing and/or diagnosing various conditions with a neurological aspect, which are not readily accurately diagnosed based on their symptoms and complex and varying physiological effects. Such conditions and diseases include without limitation Alzheimer's disease, multiple schlerosis, mental conditions including depressive disorders, bipolar disorder and schizophrenic disorders, Parkinsons' disease, epilepsy, migraine, Vascular Dementia (VaD), Fronto-temperal dementia, Lewy bodies dementia, Creutzfeld-Jacob disease and vCJD (“mad cow” disease).

As appears from the description herein, the present invention is particularly suited for using biosignal data obtained by electroencephalographic (EEG) measurements. However, other multidimensional biosignal measurement techniques used in neurophysiologic studies may as well be used in the methods of the invention, separately or in a combination of one or more techniques. Such techniques include without limitation magnetic resonance imaging (MRI), functional magnetic resonance imaging (FMRI), magnetoencephalographic (MEG) measurements, positron emission tomography (PET), CAT scanning (Computed Axial Tomography) and single photon emission computerized tomography (SPECT). When referring to ‘one biosignal measurement’ in the context herein, what is meant is a measurement of biosignal data with one technique such as any of the above, i.e. ‘one biosignal measurement’ used in the method herein will give rise to multidimensional data. In all cases it is implied that the data is computerized, e.g. images digitized and so forth.

In an embodiment of the invention the method comprises subjecting the subject to a sensory and/or physiologic stimulus prior to or during the biosignal measurement, which can comprise evoked auditory potentials, monotone modulation, photic stimulation, visual evoked potentials, eyes open/eyes closed transition, concentration (e.g. performing a mental task, listening to music, watching pretty pictures etc.), hyperventilation, rectal stimulation and the like.

In a related aspect the invention provides a method for assessing and/or diagnosing a neurological condition such as any of the above mentioned, which method comprises performing a biosignal measurement as described above on a subject which has been administered a probe compound as defined above, and analyzing the obtained multidimensional data to determine the presence of a discriminatory (diagnostic) pattern (signal) that has been previously determined as described above, which pattern/signal would indicate that the subject is afflicted with or has a predisposition for said neurological condition.

The biosignal measurement and selection of probe compound in the diagnostic method essentially matches the measurement and compound used to determine the discriminatory pattern. For example, if the discriminatory pattern is based on biosignal data obtained both prior to and after administration of the probe compound the diagnostic method will comprise such analogous pre- and post-administration measurements. Also, if the data in the pattern determination comprises measurements during sensory stimulus such as mentioned above, analogous measurements are included in the diagnostic method.

### Multidimensional Pattern Analysis

The invention relies on state of the art multidimensional pattern analysis techniques that are used in order to analyze complex biosignal data obtained in accordance with the invention in order to generate a discriminatory signal for a condition and also to use discriminatory signal(s) obtained thereby to assess and/or diagnose said condition. For a review of pattern analysis and classification see, e.g., Duda et al. “Pattern Classification”, John Wiley & Sons, Inc. (2001).

In order to generate a discriminatory signal (i.e. a “classifier”) that can be used to classify a subject in one of two (or potentially more) groups such as, e.g., group I: subjects with condition X, and group II: subjects not afflicted with condition X, one needs a training dataset comprising at least one dataset from one or more known subjects from each group (i.e. it is known to which group the subjects belong).

Potential features are identified and screened in various combinations in order to generate a classifier. The classifier is generally tested with data from pre-classified subjects to see if the classification is reliable. If the classifier is determined to be reliable, it can be used to classify unknown subjects.

Various pattern classification schemes can be used in the methods of the invention such as, e.g., k-NN nearest neighbor scheme (k-NN), support vector machines (SVM), Linear discriminant analysis, quadratic discriminant analysis, regularized discriminant analysis, Logistic regression, naive Bayes classifier, hidden Markov model classifiers, neural network based classifiers including multi-layer perception networks and radial basis function networks, support vector machines (SVM), Least-squares SVM, Classification Tree-based classifiers including Classification and Regression Trees (CART), ID3, C4.5, C5.0, AdaBoost, and ArcX4, Tree-based ensemble classifier including Random Forests™.

The k-NN scheme is particularly well suited for classification of small data sets while SVM performs in general better on larger sets and is known to possess remarkable generalization properties.

Example 1 illustrates in greater detail how a classifier (i.e. discriminatory pattern) is obtained from EEG data for Alzheimer's disease (AD). In the example the substance scopolamine is used as a probe compound to perturb biophysical pathways affecting the measured signals and biosignals (EEG) are recorded both before and after the probe compound administration in a known group of AD patients and a control group with individuals not afflicted with AD (determined by clinical evaluation). A classifier is generated and shown to be reliable and can consequently be used to diagnose AD in unknown subjects.

Preferably, a set of classifiers (e.g. feature pair classifiers such as described in Example 1) are combined in an ensemble which subsequently is used as the determining classifier for classifying unknowns. Such methods are well known in the art, for example ensamble classifiers.

While Example 1 describes in detail one embodiment of the invention, it will be appreciated that alternative embodiments can be configured and optimized, e.g. to better suit the diagnosis of other diseases and the use of other measurement techniques in addition to or as an alternative to EEG.

As shown in Example 1, features from the data are often selected based on prior knowledge, e.g. known variables of interest extracted from the raw data. In the example, none of the selected features from the EEG data by themselves give a clear positive indication for the disease which is assessed. By administering a suitable probe compound and looking specifically at the changes of the selected features before and after administration such as e.g. by calculating the ratio or difference for each feature before and after the probe administration (F₁^{post}/F₁^{pre} or F₁^{post}−F₁^{pre}, etc.) reliable classifiers can be generated.

In the case of EEG data, several known variables can be selected for initial analysis which include for example the 16 features listed in Example 1, either measured only after the probe administration or preferably both before and after the administration.

Additional features can be generated by using sensory stimuli such as those mentioned above, which could be one or more of the above mentioned features measured while the subject is subjected to the stimulus. Thus, one EEG variable F₁ can generate a set of features: F₁^{pre}, F₁^{post} without stimulus S₁ and F₁^{pre}, F₁^{post} with stimulus in addition to ratios and differences of these different F₁ features.

As mentioned above, the invention further relates to a system as described above for assessing a neurological condition such as any of the above mentioned, the system comprising a receiver unit 11 where the receiver unit is preferably adapted to convert received biosignal (could e.g. be an image) to digital signal, a computer 13 with memory 4 for storing recorded biosignals from a subject 8 and a programmable memory 5 for storing a program, and a processor 6 for executing the instructions encoded in said program for analyzing said signals subject to said instructions, wherein the computer in accordance with the instructions encoded in said program performs a pattern recognition analysis on at least a subset of said recorded signals to obtain a pattern in accordance with the invention, and compares the obtained pattern with a reference pattern template previously determined as described herein to classify said subject, i.e. to indicate whether the subject suffers from or has a predisposition for said neurological condition.

The term ‘pattern’ refers to a selected set of features generated from the recorded signals such as described above.

As shown in FIG. 11, the hardware components of the system of the invention will generally comprise conventional personal computer 13 and electronic components well known in the art, e.g. a receiver 9 specially configured to receive EEG signals and/or other biosignals. A regular personal computer can be used for storing and operating the program for performing the data analysis as well as for storing the recorded biosignals and control signal/pattern database.

In a preferred embodiment of the invention, the system and methods of the invention comprise a “self-learning” system comprising a reference database on which the reference pattern is based and wherein data for each new subject that is positively classified with the system is added to the database to make the classifier even more reliable.

FIG. 12 shows a block diagram of the classification ensemble. The selection algorithm f (14) receives the output from multiple pattern classifiers (15), each analyzing different feature pairs, and generates the output x which represents the overall likelihood.

Certain specific details of the disclosed embodiment are set forth for purposes of explanation rather than limitation, so as to provide a clear and thorough understanding of the present invention. However, it should be understood by those skilled in this art, that the present invention might be practiced in other embodiments that do not conform exactly to the details set forth herein, without departing significantly from the spirit and scope of this disclosure. Further, in this context, and for the purposes of brevity and clarity, detailed descriptions of well-known apparatuses, circuits and methodologies have been omitted so as to avoid unnecessary detail and possible confusion.

Reference signs are included in the claims, however the inclusion of the reference signs is only for clarity reasons and should not be construed as limiting the scope of the claims.

### REFERENCES

Adler G. et al. EEG coherence In Alzheimer's dementia. Journal of neural transmission, Vol 110, no. 9, 2003.
Babiloni, C. et al. Mapping distributed sources of cortical rhythms in mild Alzheimer's disease. A multicentric EEG study. Neuroimage 22 57-67, 2004.
Babiloni, C. et al. Abnormal fronto-parietal coupling of brain rhythms in mild Alzheimer's disease: a multicentric EEG study. European Journal of Neuroscience, Vol. 19, pp. 2583-2590 2004.
Bennys, K. et al. Diagnostic value of quantitative EEG in Alzheimer's disease. Neurophysiol Clin, 31, 153-60, 2001.
Benvenuto J. et al. Identification of diagnostic evoked response potential segments in Alzheimer's disease. Experimental Neurology 176, 269-276, 2002.
Brunovsky M., Matousek M. et al. EEG assessment of the degree of dementia. Neuropsychobiology 48:19-26, 2003.
Cichocki et al. EEG filtering based on blind source separation (BSS) for early detection of Alzheimer's disease. Clinical Neurophysiology, In-press, 2004.
Cho, S. Y. et al. Automatic recognition of Alzheimer's disease using genetic algorithms and neural network. Lecture Notes in Computer Science, Springer-Verlag, Volume 2658, pp. 695-702, 2003.
Claus, J. J. et al. The diagnostic value of electroencephalography in mild senile Alzheimer's disease. Clinical Neurophysiology 110, 825-832, 1999.
Duda R O, Hart P E and Stork D G, Pattern Classification, Wiley-Interscience; 2nd edition, 2000.
Hara J. et al. Cortical atrophy in Alzheimer's disease unmasks electrically silent sulci and lowers EEG dipolarity. IEEE TBME 46, 8, 1999.
Holschneider, D. P. et al. Loss of high-frequency brain electrical response to thiopental administration in Alzheimer's-type dementia. Neuropsychopharmacology 16, 4, 1997.
Holschneider, D. P. et al. Attenuation of brain high frequency electrocortical response after thiopental in early stages of Alzheimer's dementia. Psychopharmacology, 149, 6-11, 2000.
Hongzhi, Q. I. et al. Mutual information entropy research on dementia EEG signals. The Fourth International Conference on Computer and Information Technology (CIT'04), Sep. 14-16, 2004, Wuhan, China.
Huang, C. et al. Discrimination of Alzheimer's disease and mild cognitive impairment by equivalent EEG sources: a cross-sectional and longitudinal study. Clinical Neurophysiology 111, 1961-1967, 2000.
Hyung-Rae K. et al. Analysis of the spatiotemporal EEG pattern in Alzheimer's disease. Journal of the Korean Physical Society, Vol. 34, No. 4, April 1999.
Jelles B. et al. Decrease of non-linear structure in the EEG of Alzheimer patients compared to healthy controls. Clinical Neurophysiology 110, 1159-1167, 1999.
Jeong, J. et al. Non-linear dynamical analysis of the EEG in Alzheimer's disease with optimal embedding dimension. Electroencephalography and clinical Neurophysiology 106, 220-228, 1998.
Jeong, J. et al. Mutual information analysis of the EEG in patients with Alzheimer's disease. Clinical Neurophysiology 112, 827-835, 2001.
Jeong, J. EEG dynamics in patients with Alzheimer's disease. Clinical Neurophysiology, In-press, 2004.
Jimenez-Escrig A. et al. P300 latency reveals cholinergic enhancement produced by rivastigmine in Alzheimer's disease. Alzheimer's Reports, Vol. 4, No. 1, 2001.
Jonkman, E. J. The role of the electroencephalogram in the diagnosis of dementia of the Alzheimer type: an attempt at technology assessment. Neurophysiol Clin, 27, 211-9, 1997.
Kikuchi, M. et al. EEG harmonic responses to photic stimulation in normal aging and Alzheimer's disease: differences in interhemispheric coherence. Clinical Neurophysiology 113, 1045-1051, 2002.
Koenig, T. et al. Decreased EEG synchronization in Alzheimer's disease and mild cognitive impairment. Neurobiology of aging, In press, 2004.
Locatelli, T. et al. EEG coherence in Alzheimer's disease. Electroencephalography and clinical Neurophysiology 106, 229-237, 1998.
Londos, E., Rosen, I. et al. Regional cerebral blood flow and EEG in clinically diagnosed dementia with Lewy bodies and Alzheimer's disease. Arch. Gerontol. Geriatr. 36, 231-245, 2003.
Montplaisir, J. et al. Sleep disturbances and EEG slowing in Alzheimer's disease. Sleep Research Online 1(4): 147-151, 1998
Moretti et al. Individual analysis of EEG frequency and band power in mild Alzheimer's disease. Clinical Neurophysiology 115, 299-308, 2004.
Musha, T. et al. A new EEG method for estimating cortical neuronal impairment that is sensitive to early stage Alzheimer's disease. Clinical Neurophysiology 113, 1052-1058, 2002.
Nikulin V., Brismar T., Long-range temporal correlation in alpha and beta oscillations: effects of arousal level and test-retest reliability. Clinical Neurophysiology 115, 1896-1908, 2004.
Pijnenburg Y. A. L. et al. EEG synchronization likelihood in mild cognitive impairment and Alzheimer's disease during a working memory task. Clinical Neurophysiology, In-press, 2004.
Pucci, E. et al. EEG spectral analysis in Alzheimer's disease and different degenerative dementias. Archives of Gerontology and Geriatrics 26, 283-297, 1998.
Pucci, E. et al. EEG power spectrum differences in early and late onset forms of Alzheimer's disease. Clinical Neurophysiology 110, 621-631, 1999.
Rodriquez, G. et al. EEG spectral profile to stage Alzheimer's disease. Clinical Neurophysiology 110, 1831-1837, 1999.
Shawe-Taylor J and Cristianini N, Kernel Methods for Pattern Analysis, Cambridge University Press, 2004.
Signorino M. et al. EEG spectral analysis in vascular and Alzheimer dementia. Electroencephalography and clinical neurophysiology 94:313-325, 1995.
Sumi, N. et al. Interpeak latency of auditory event-related potentials (P300) in senile depression and dementia of the Alzheimer type. Psychiatry and Clinical Neurosciences, 54, 679-684, 2000.
Stam C. J. et al. EEG synchronization in mild cognitive impairment and Alzheimer's disease. Acta Neurol Scand 108, 90-96, 2003.
Stam C. J. et al. Disturbed fluctuations of resting state EEG synchronization in Alzheimer's disease. Clinical Neurophysiology, In-press, 2004.
Stevens A. et al. Cognitive decline unlike normal aging is associated with alterations of EEG temporo-spatial characteristics. Eur Arch Psychiatry Clin Neurosci 248:259-266, 1998.
Stevens A. et al. Dynamic regulation of EEG power and coherence is lost early and globally in probable DAT. Eur Arch Psychiatry Clin Neurosci 251:199-204, 2001.
Strik, W. K. et al. Decreased EEG microstate duration and anteriorisation of the brain electrical fields in mild and moderate dementia of the Alzheimer type. Psychiatry Research: NeuroImaging Section, 75, 183-191 (1997).
Vesna J. et al. Quantitative electroencephalography in mild cognitive impairment: longitudinal changes and possible prediction of Alzheimer's disease. Neurobiology of aging 21, 533-540, 2000.
Wada Y. et al. Abnormal functional connectivity in Alzheimer's disease: intrahemisphereic EEG coherence during rest and photic stimulation. Eur Arch Psychiatry Clin Neurosci 248:203-208, 1998.

1. A method of generating a discriminatory signal for a neurological condition comprising:
providing at least one probe compound that has a neurophysiologic effect,
obtaining biosignal data from a subject based on biosignal measurements obtained from biosignal measuring device adapted for placement on a subject, wherein said biosignal data are obtained posterior to the administering of said probe compound to the subject,
providing analogous biosignal reference data for reference subjects in at least one reference group posterior to the administering of said probe compound, wherein each group represents reference subjects having at least one common characteristics, wherein the reference data are utilized for:
performing a pre-scanning on the reference data for each reference subject within the same group for identifying correlations between the reference data for the reference subjects within the same group, the identified correlations being used as a criteria for defining reference features fi, iε{1, . . . , N_{f}} for the reference subjects within said same group,
determining reference feature values for said reference features fi for each respective reference subject,
defining a domain Vε{fᵢ₁, fᵢ₂, . . . , f_{iN}} containing domain elements fᵢ₁, fᵢ₂, . . . , f_{iN}, each domain element fᵢₘ being a combination of two or more of said reference features and defines a two or more dimensional feature space,
determining, for each respective feature space as defined by said domain element fᵢₘ, the distribution of the reference feature values for each respective reference subjects,
implementing the distribution of the reference feature values of said reference subjects for determining a posterior probability vector P^{ref}=[p(fᵢ₁), p(fᵢ₂), . . . , p(f_{iN})] for each respective reference subject wherein each respective element p(fᵢₘ) of the posterior probability vector P indicates the probability that the reference subject belongs to said group in terms of said domain elements,
applying a filtering process on said posterior probability vectors, said filtering process being based on removing those vectors or vector elements that are above or below a pre-defined threshold value, the remaining vectors or vector elements being implemented for constructing a reference distribution for said reference subjects as a function of said domain elements; and
utilizing said biosignal data from said subject for calculating identical posterior probability vector P^{subj}=[p(fᵢ₁), p(fᵢ₂), . . . , p(f_{iN})] for said subject,
wherein said discriminatory signal is generated based on comparison between said posterior probability vector for said subject P_{subj}=[p(fᵢ₁), p(fᵢ₂), . . . , p(f_{iN})] and the distribution of said features or feature combinations.

2-24. (canceled)

25. The method of claim 1, further comprising obtaining biosignal data from said subject and said reference subjects prior to administering said probe compound.

26. The method of claim 1, further comprising selecting out only those elements in said reference posterior probability vectors that have variance value above a pre-defined threshold value.

27. The method of claim 1 wherein said one or more biosignal measurements comprise an electroencephalographic (EEG) measurement.

28. The method of claim 1, wherein the neurological condition is selected from the group consisting of Alzheimer's disease, multiple schlerosis, mental conditions including depressive disorders, bipolar disorder and schizophrenic disorders, Parkinsons' disease, epilepsy, migraine, Vascular Dementia (VaD), Fronto-temporal dementia, Lewy bodies dementia, Creutzfeld-Jacob disease, and vCJD (“mad cow” disease).

29. The method of claim 1, wherein said one or more biosignal measurements comprise a biosignal measurement selected from the group consisting of magnetic resonance imaging (MRI), functional magnetic resonance imaging (FMRI), magnetoencephalographic (MEG) measurements, positron emission tomography (PET), CAT scanning (Computed Axial Tomography), and single photon emission computerized tomography (SPECT).

30. The method of claim 1, wherein said at least one probe compound is selected from the group consisting of GABA affecting drugs, propofol, etomidate, barbiturates, methohexital, thiopental, thiamylal, buthalital, thialbarbital, hexobarbital, pentobarbital, secobarbital, hexethal, butalbital, cyclobarbital, talbutal, phenobarbital, mephobarbital, barbital, benzodiazepines, alprazolam, bromazepam, chlordiazepoxide, clobazam, clonazepam, clorazepate, clozapine, olanazapine diazepam, estazolam, flunitrazepam, flurazepam, halazepam, ketazolam, loprazolam, lorazepam, lormetazepam, medazepam, midazolam, nitrazepam, nordazepam, oxazepam, prazepam, ouazepam, temazepam, and triazolam, cholinergic agonists, aceclidine, AF-30, AF150, AF267B, alvameline, arecoline, bethanechol, CDD-0102, CDD-0034-C, CDD-0097-A, cevimeline, CI 1017, cis-dioxolane, milameline, muscarine, oxotremorine, pilocarpine, RS86, RU 35963, RU 47213, sabcomeline, SDZ-210-086, SR 46559A, talsaclidine, tazomeline, UH5, xanomeline, and YM 796, cholinergic antagonists, AF-DX 116, anisotropine, aprophen, AQ-RA 741, atropin, belladonna, benactyzine, benztropine, BIBN 99, DIBD, cisapride, clidinium, darifenacin, dicyclomine, glycopyrrolate, homatropine, atropine, hyoscyamine, ipratropium, mepenzolate, methantheline, methscopolamine, PG-9, pirenzepine, propantheline, SCH-57790; SCH-72788, SCH-217443, scopolamine, tiotropium, tolterodine, and trihexyphenidyl, acetyl choline esterase (ACE) inhibitors, 4-aminopyridine, 7-methoxytacrine, amiridine, besipirdine, CHF2819, CI-1002, DMP 543, donepezil, eptastigmine, galantamine, huperzine A, huprine X, huprine Y, MDL 73745, metrifonate, P10358, P11012, phenserine, physostigmine, ouilostigmine, rivastigmine, Ro 46-5934, SM-10888, suronacrine, T-82, tacrine, TAK-147, tolserine, trifluoroacetophenone, TV3326, velnacrine, zifrosilone, ACh release enhancers, linopirdine, XE991, Choline uptake enhancers, MKC-231, Z-4105, nicotinic agonists, ABT-089, ABT-418, GTS-21, SIB-1553A, NMDA antagonists, ketamine, memantine, serotonin inhibitors, cinanserin hydrochloride, fenclonine, fonazine mesylate, xylamidine tosylate, serotonin antagonists, altanserin tartrate, aAmesergide, cyproheptadiene, granisetron, homochlorcyclizine, ketanserin, mescaline, mianserin, mirtazapine, perlapine, pizotyline, olanzapine, ondansetron, oxetorone, risperidone, ritanserin, tropanserin hydrochloride, zatosetron, serotonin agonists, 2-methylserotonin, 8-hydroxy-DPAT, buspirone, gepirone, ipsapirone, rizatriptan, sumatriptan, zolmatriptan, serotonin reuptake inhibitors, citalopram, escitalopram oxalate, fluoxetine, fluvoxamine, paroxetine, sertraline, dopamine antagonists, pimozide, ouetiapine, metoclopramide, dopamine precursors, and levodopa.

31. The method of claim 1, wherein said one or more biosignal measurements comprise an electroencephalographic (EEG) measurement.

32. The method of claim 1, wherein two or more compounds are used for stimulating two or more different neurophysiologic effects.

33. The method of claim 1, further comprising subjecting the subject to a sensory stimulus prior to or during the biosignal measurement.

34. The method of claim 1, wherein said features are selected from a group consisting of the absolute delta power, the absolute theta power, the absolute alpha power, the absolute beta power, the absolute gamma power, the relative delta power, the relative theta power, the relative alpha power, the relative beta power, the relative gamma power, the total power, the peak frequency, the median frequency, the spectral entropy, the DFA scaling exponent (alpha band oscillations), the DFA scaling exponent (beta band oscillations), and the total entropy.

35. A computer readable media for storing instructions for enabling a processing unit to execute the method steps in claim 1.

36. A system adapted for generating a discriminatory signal for a neurological condition of a subject posterior to administering at least one compound that has a neurophysiologic effect comprising:
a receiver unit for receiving biosignal data for a subject from biosignal measuring device after administering said at least one compound,
an internal or external storage means for storing analogous biosignal reference data for reference subjects in at least one reference group posterior to the administering of said probe compound,
a processor for utilizing the reference data for generating reference distribution by means of:
performing a pre-scanning on the reference data for each reference subject within the same group for identifying correlations between the reference data for the reference subjects within the same group, the identified correlations being used as a criteria for defining reference features fi, iε{1, . . . , N_{f}} for the reference subjects within said same group,
determining reference feature values for said reference features fi for each respective reference subject,
defining a domain Vε{fᵢ₁, fᵢ₂, . . . , f_{iN}} containing domain elements fᵢ₁, fᵢ₂, . . . , f_{iN}, each domain element fᵢₘ being a combination of two or more of said reference features and defines a two or more dimensional feature space,
determining, for each respective feature space as defined by said domain element fᵢₘ, the distribution of the reference feature values for each respective reference subjects, implementing the distribution of the reference feature values of said reference subjects for determining a posterior probability vector P^{ref}=[p(fᵢ₁), p(fᵢ₂), . . . , p(f_{iN})] for each respective reference subject wherein each respective element p(fᵢₘ) of the posterior probability vector P indicates the probability that the reference subject belongs to said group in terms of said domain elements,
applying a filtering process on said posterior probability vectors, said filtering process being based on removing those vectors or vector elements that are above or below a pre-defined threshold value, the remaining vectors or vector elements being implemented for constructing a reference distribution for said reference subjects as a function of said domain elements, and
a processor for utilizing said biosignal data from said subject for calculating identical posterior probability vector P^{subj}=[p(fᵢ₁), p(fᵢ₂), . . . , p(f_{iN})] for said subject, said processor being adapted for generating said discriminatory signal based on comparison between said posterior probability vector for said subject and said reference distribution.

37. A method of using a probe to diagnose a neurological condition comprising:
providing a probe that comprises a GABA affecting drug, propofol, etomidate, barbiturates, methohexital, thiopental, thiamylal, buthalital, thialbarbital, hexobarbital, pentobarbital, secobarbital, hexethal, butalbital, cyclobarbital, talbutal, phenobarbital, mephobarbital, barbital, benzodiazepine, alprazolam, bromazepam, chlordiazepoxide, clobazam, clonazepam, clorazepate, clozapine, olanazapine diazepam, estazolam, flunitrazepam, flurazepam, halazepam, ketazolam, loprazolam, lorazepam, lormetazepam, medazepam, midazolam, nitrazepam, nordazepam, oxazepam, prazepam, ouazepam, temazepam, and triazolam, cholinergic agonist, aceclidine, AF-30, AF150, AF267B, alvameline, arecoline, bethanechol, CDD-0102, CDD-0034-C, CDD-0097-A, cevimeline, CI 1017, cis-dioxolane, milameline, muscarine, oxotremorine, pilocarpine, RS86, RU 35963, RU 47213, sabcomeline, SDZ-210-086, SR 46559A, talsaclidine, tazomeline, UH5, xanomeline, and YM 796, cholinergic antagonist, AF-DX 116, anisotropine, aprophen, AQ-RA 741, atropin, belladonna, benactyzine, benztropine, BIBN 99, DIBD, cisapride, clidinium, darifenacin, dicyclomine, glycopyrrolate, homatropine, atropine, hyoscyamine, ipratropium, mepenzolate, methantheline, methscopolamine, PG-9, pirenzepine, propantheline, SCH-57790; SCH-72788, SCH-217443, scopolamine, tiotropium, tolterodine, and trihexyphenidyl, acetyl choline esterase (ACE) inhibitors, 4-aminopyridine, 7-methoxytacrine, amiridine, besipirdine, CHF2819, CI-1002, DMP 543, donepezil, eptastigmine, galantamine, huperzine A, huprine X, huprine Y, MDL 73745, metrifonate, P10358, P11012, phenserine, physostigmine, ouilostigmine, rivastigmine, Ro 46-5934, SM-10888, suronacrine, T-82, tacrine, TAK-147, tolserine, trifluoroacetophenone, TV3326, velnacrine, zifrosilone, ACh release enhancer, linopirdine, XE991, Choline uptake enhancer, MKC-231, Z-4105, nicotinic agonist, ABT-089, ABT-418, GTS-21, SIB-1553A, NMDA antagonist, ketamine, memantine, serotonin inhibitors, cinanserin hydrochloride, fenclonine, fonazine mesylate, xylamidine tosylate, serotonin antagonists, altanserin tartrate, aAmesergide, cyproheptadiene, granisetron, homochlorcyclizine, ketanserin, mescaline, mianserin, mirtazapine, perlapine, pizotyline, olanzapine, ondansetron, oxetorone, risperidone, ritanserin, tropanserin hydrochloride, zatosetron, serotonin agonists, 2-methylserotonin, 8-hydroxy-DPAT, buspirone, gepirone, ipsapirone, rizatriptan, sumatriptan, zolmatriptan, serotonin reuptake inhibitor, citalopram, escitalopram oxalate, fluoxetine, fluvoxamine, paroxetine, sertraline, dopamine antagonist, pimozide, ouetiapine, metoclopramide, dopamine precursor, and levodopa; and
performing the method of claim 1 using said probe.

38. A method of initiating a neurological reaction for dementia of the Alzheimer's type (AD group) comprising:
providing a probe that contains scopolamine; and
performing the method of claim 1 using said probe.

39. A method of using software to compare data measured on a control subject with data measured on a subject suspected to suffer from a neurological condition, comprising:
using received biosignal data obtained from biosignal measuring device for determining one or more features, said biosignal data being obtained after administering said at least one compound,
calculating posterior probability vector for said subject in accordance to posterior probability vectors obtained from reference subjects from at least one group, said posterior probability vectors consisting of probability values associated to feature or a feature combination elements determined from biosignal data for said reference subjects, said posterior probability vectors resulting in a statistical distribution of said features or feature combinations for said reference subjects, and
comparing the posterior probability vector for said subject with a distribution.

40. A method for assessing a neurological condition in a subject comprising:
administering to the subject a probe compound that has a neurophysiological effect;
performing one or more biosignal measurements on the subject to obtain multidimensional biosignal data; and
analyzing said multidimensional biosignal data with multidimensional analytical techniques to determine the presence of a discriminatory pattern which indicates that the subject is afflicted with or has a predisposition for said neurological condition.

41. The method of claim 40, wherein said one or more biosignal measurements on the subject which comprise an electroencephalographic measurement.

42. The method of claim 40, wherein biosignal measurements are performed both prior to and after said administration of said probe compound.

43. The method of claim 40, wherein the neurological condition is selected from the group consisting of Alzheimer's disease, multiple schlerosis, mental conditions including depressive disorders, bipolar disorder and schizophrenic disorders, Parkinsons' disease, epilepsy, migraine, Vascular Dementia (VaD), Fronto-temporal dementia, Lewy bodies dementia, Creutzfeld-Jacob disease and vCJD (“mad cow” disease).

44. The method of claim 40, wherein said one or more biosignal measurements comprise a biosignal measurement selected from the group consisting of magnetic resonance imaging (MRI), functional magnetic resonance imaging (FMRI), magnetoencephalographic (MEG) measurements, positron emission tomography (PET), CAT scanning (Computed Axial Tomography) and single photon emission computerized tomography (SPECT).

45. The method of claim 40, wherein said at least one probe compound is selected from the group consisting of:
GABA affecting drugs, propofol, etomidate, barbiturates, methohexital, thiopental, thiamylal, buthalital, thialbarbital, hexobarbital, pentobarbital, secobarbital, hexethal, butalbital, cyclobarbital, talbutal, phenobarbital, mephobarbital, barbital, benzodiazepines, alprazolam, bromazepam, chlordiazepoxide, clobazam, clonazepam, clorazepate, clozapine, olanazapine diazepam, estazolam, flunitrazepam, flurazepam, halazepam, ketazolam, loprazolam, lorazepam, lormetazepam, medazepam, midazolam, nitrazepam, nordazepam, oxazepam, prazepam, ouazepam, temazepam, and triazolam, cholinergic agonists, aceclidine, AF-30, AF150, AF267B, alvameline, arecoline, bethanechol, CDD-0102, CDD-0034-C, CDD-0097-A, cevimeline, CI 1017, cis-dioxolane, milameline, muscarine, oxotremorine, pilocarpine, RS86, RU 35963, RU 47213, sabcomeline, SDZ-210-086, SR 46559A, talsaclidine, tazomeline, UH5, xanomeline, and YM 796, cholinergic antagonists, AF-DX 116, anisotropine, aprophen, AQ-RA 741, atropin, belladonna, benactyzine, benztropine, BIBN 99, DIBD, cisapride, clidinium, darifenacin, dicyclomine, glycopyrrolate, homatropine, atropine, hyoscyamine, ipratropium, mepenzolate, methantheline, methscopolamine, PG-9, pirenzepine, propantheline, SCH-57790; SCH-72788, SCH-217443, scopolamine, tiotropium, tolterodine, and trihexyphenidyl, acetyl choline esterase (ACE) inhibitors, 4-aminopyridine, 7-methoxytacrine, amiridine, besipirdine, CHF2819, CI-1002, DMP 543, donepezil, eptastigmine, galantamine, huperzine A, huprine X, huprine Y, MDL 73745, metrifonate, P10358, P11012, phenserine, physostigmine, ouilostigmine, rivastigmine, Ro 46-5934, SM-10888, suronacrine, T-82, tacrine, TAK-147, tolserine, trifluoroacetophenone, TV3326, velnacrine, zifrosilone, ACh release enhancers, linopirdine, XE991, Choline uptake enhancers, MKC-231, Z-4105, nicotinic agonists, ABT-089, ABT-418, GTS-21, SIB-1553A, NMDA antagonists, ketamine, memantine, serotonin inhibitors, cinanserin hydrochloride, fenclonine, fonazine mesylate, xylamidine tosylate, serotonin antagonists, altanserin tartrate, aAmesergide, cyproheptadiene, granisetron, homochlorcyclizine, ketanserin, mescaline, mianserin, mirtazapine, perlapine, pizotyline, olanzapine, ondansetron, oxetorone, risperidone, ritanserin, tropanserin hydrochloride, zatosetron, serotonin agonists, 2-methylserotonin, 8-hydroxy-DPAT, buspirone, gepirone, ipsapirone, rizatriptan, sumatriptan, zolmatriptan, serotonin reuptake inhibitors, citalopram, escitalopram oxalate, fluoxetine, fluvoxamine, paroxetine, sertraline, dopamine antagonists, pimozide, ouetiapine, metoclopramide, dopamine precursors, and levodopa.

46. The method of claim 40, further comprising subjecting the subject to a sensory stimulus prior to or during the electroencephalographic measurement.

47. The method of claim 32, wherein said discriminatory pattern is obtained by the method of claim 1.
